(19) United States
(12) Patent Application Publication
(10) Pub. No.: US 2009/0247577 A1
(43) Pub. Date: Oct. 1, 2009

(54) PYRROLIDINE DERIVATIVES USEFUL AS BACE INHIBITORS

(76) Inventors: Olivier Rogel, Hesingue (FR); Jean-Michel Rondeau, Rixheim (FR); Heinrich Rueeger, Fueh (CH); Oliver Simic, Basel (CH); Finton Sirockin, St. Louis (FR); Marina Tintelnot-Blomley, Maulburg (DE)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/303,495

(22) PCT Filed: Jun. 4, 2007
(86) PCT No.: PCT/EP2007/004959

§ 371 Date: Dec. 4, 2008

(30) Foreign Application Priority Data
Jun. 5, 2006 (GB) 0611064.7

(51) Int. Cl. A61K 31/4709; C07D 401/12

(52) U.S. Cl. 514/312; 546/156

(57) ABSTRACT

Novel 3-mono-, 3,4-di- and 3,4,4,-tri-substituted pyrrolidine compounds, these compounds for use in the diagnostic and therapeutic treatment of a warm-blooded animal, especially for the treatment of a disease (disorder) that depends on the activity of beta-secretase and/or the generation of beta-amyloid and the subsequent aggregation into oligomers and fibrils; the use of a compound of that class for the preparation of a pharmaceutical formulation for the treatment of a disease that depends on the activity of beta-secretase and/or the generation of beta-amyloid and the subsequent aggregation into oligomers and fibrils; pharmaceutical formulations comprising a said substituted pyrrolidine compound, and/or a method of treatment comprising administering a said substituted pyrrolidine compound. The substituted pyrrolidine compounds are especially of the formula I,

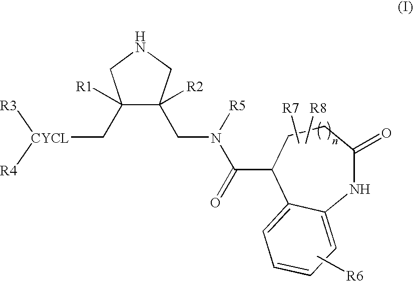

wherein the substituents are as defined in the specification.

The invention relates to (3,4-di-, 3,4,4-tri- or 3,3,4,4-tetra-)substituted pyrrolidine compounds, these compounds for use in the diagnostic and therapeutic treatment of a warm-blooded animal, especially for the treatment of a disease (=disorder) that depends on the activity of beta-secretase and the generation of beta-amyloid and the subsequent aggregation into oligomers and fibrils; the use of a compound of that class for the preparation of a pharmaceutical formulation for the treatment of a disease that depends on the activity of beta-secretase and the generation of beta-amyloid and the subsequent aggregation into oligomers and fibrils; the use of a compound of that class in the treatment of a disease that depends on the activity of beta-secretase and the generation of beta-amyloid and the subsequent aggregation into oligomers and fibrils; pharmaceutical formulations comprising said substituted pyrrolidine compound, and/or a method of treatment comprising administering said substituted pyrrolidine compound, a method for the manufacture of said substituted pyrrolidine compound, and novel intermediates and partial steps for its synthesis.

The present invention provides especially compounds of the formula I,

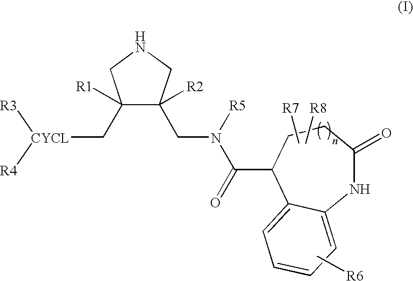

wherein
R¹ and R² are independently of each other hydrogen, C₁-C₇-alkoxy or halogen;
CYCL is aryl or cycloalkyl;
R³ and R⁴ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, amino, mono- or di-(C₁-C₇-alkyl)-amino, C₁-C₇-alkanoylamino, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano;
R⁵ is unsubstituted or substituted alkyl, unsubstituted or substituted aryl, substituted or unsubstituted alkenyl, unsubstituted or substituted mono- or bicyclic heterocyclyl, unsubstituted or substituted cycloalkyl, unsubstituted or substituted aryl-alkyl, unsubstituted or substituted mono- or bicyclic heterocyclyl-alkyl or unsubstituted or substituted cycloalkyl-alkyl;
n is 0 or 1;
R⁶, R⁷ and R⁸ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, amino, mono- or di-(C₁-C₇-alkyl)-amino, C₁-C₇-alkanoylamino, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano; or when R⁷ and R⁸ are both C₁-C₇-alkyl, they may form a C₃-C₇-cycloalkyl ring; or a salt thereof.

The agents of the invention are inhibitors of aspartic proteases and can be used for the treatment of disorders involving processing by such enzymes. Particularly they inhibit beta-secretase and as such inhibit the generation of beta-amyloid and the subsequent aggregation into oligomers and fibrils.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo. If not explicitly or implicitly stated otherwise, halo can also stand for more than one halogen substitutent in moieties such as alkyl, alkanoyl and the like (e.g. in trifluoromethyl, trifluoroacetyl).

Unsubstituted or substituted aryl preferably is a is mono- or polycyclic, especially monocyclic, bicyclic, tricyclic aryl with 6 to 22 carbon atoms, especially phenyl, naphthyl, indenyl or fluorenyl, and is unsubstituted or substituted by one or more, especially one to three, moieties, preferably independently selected from the group consisting of a substitutent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇ alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;

from C₂-C₇-alkenyl, C₂-C₇-alkinyl, phenyl, naphtyl, heterocyclyl, especially as defined below for heterocyclyl, preferably selected from pyrrolyl, furanyl, thienyl, pyrimidine-2,4-dione-1-, -3- or -5-yl and benzo[1,3]-dioxolyl, phenyl- or naphthyl- or heterocyclyl-C₁-C₇-alkyl wherein heterocyclyl is as defined below, preferably selected from pyrrolyl, furanyl, thienyl and benzo[1,3]-dioxolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, halo-C₁-C₇-alkoxycarbonyl, phenyl- or naphthyloxycarbonyl, phenyl- or naphthyl-C₁-C₇-alkoxycarbonyl, (N,N—) di-(C₁-C₇-alkyl)-amino-C₁-C₇-alkoxycarbonyl, carbamoyl, N-mono or N,N-di-(naphthyl- or phenyl-)-aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkinylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N—) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl.

Unsubstituted or substituted heterocyclyl is a mono- or bicyclic or if not part of a substituent R¹ or R² or if not a substituent R¹ and R² further polycyclic heterocyclic moiety (meaning that in cases where unsubstituted or substituted heterocyclyl is part of a substituent R¹ and R² (e.g. in heterocyclylalkyl) or itself is a moiety R¹ or R², it comprises not more than two rings annelated to each other, while in the case of substitutents R³ comprising or consisting of unsubstituted or substituted heterocyclyl it may comprise more than two rings annelated to each other), preferably a mono- or bicyclic or, if not part of a substituent R¹ or R² or if not a substituent R¹ and R², mono-, bi- or further tricyclic-, (in all cases mono-cyclic or annelated systems mentioned so far) unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, S(═O)— or S—(═O)₂—) which is unsubstituted or substituted by one or more, e.g. up to three, substitutents preferably independently selected from the substitutents mentioned above for aryl and from oxo. Preferably, unsubstituted or substituted heterocyclyl is selected from the following moieties:

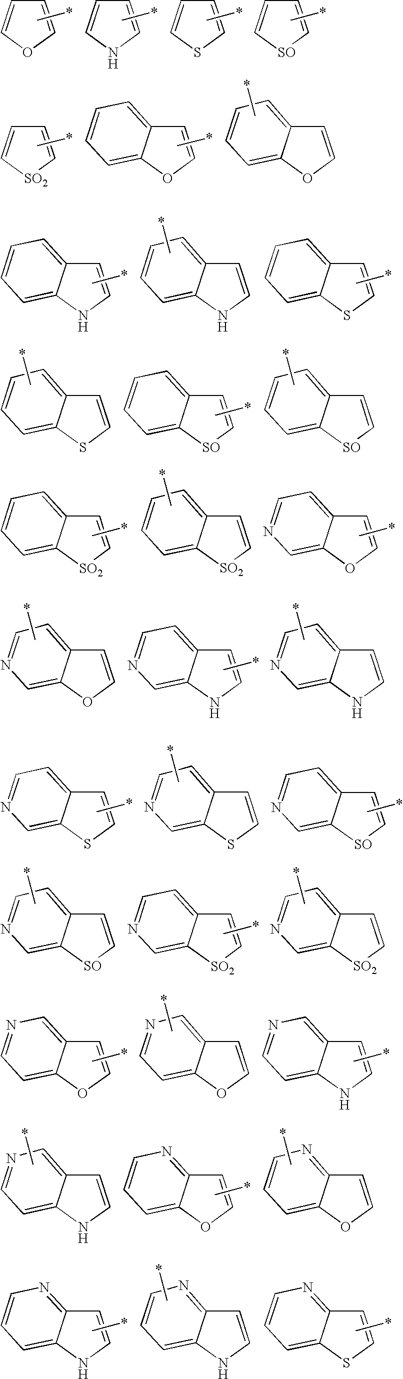

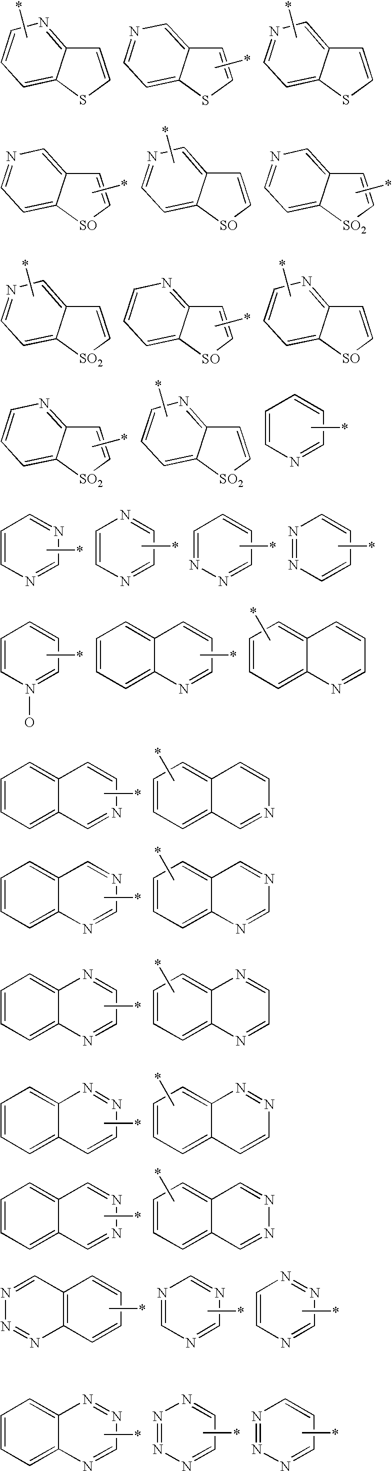

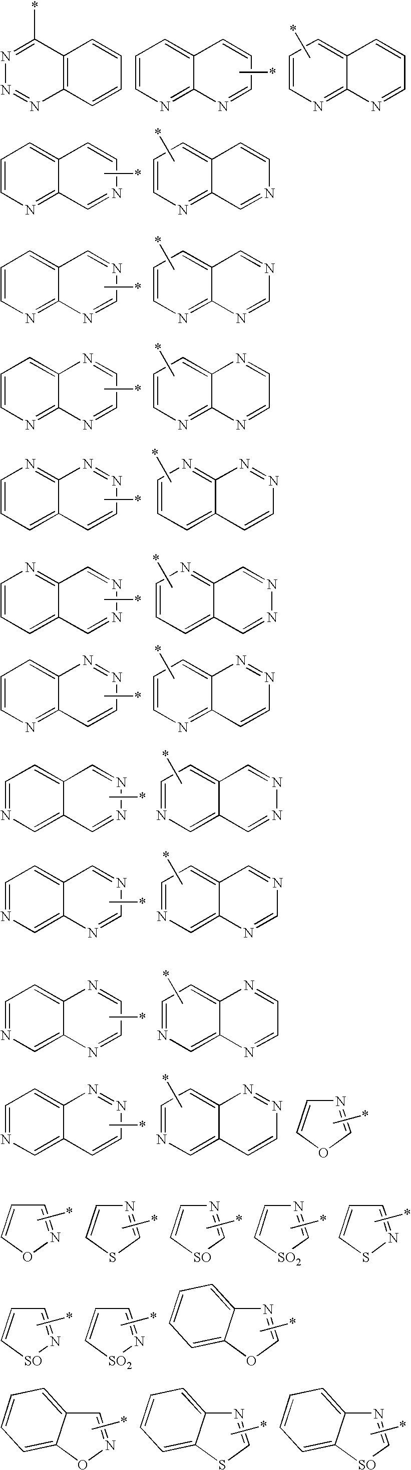

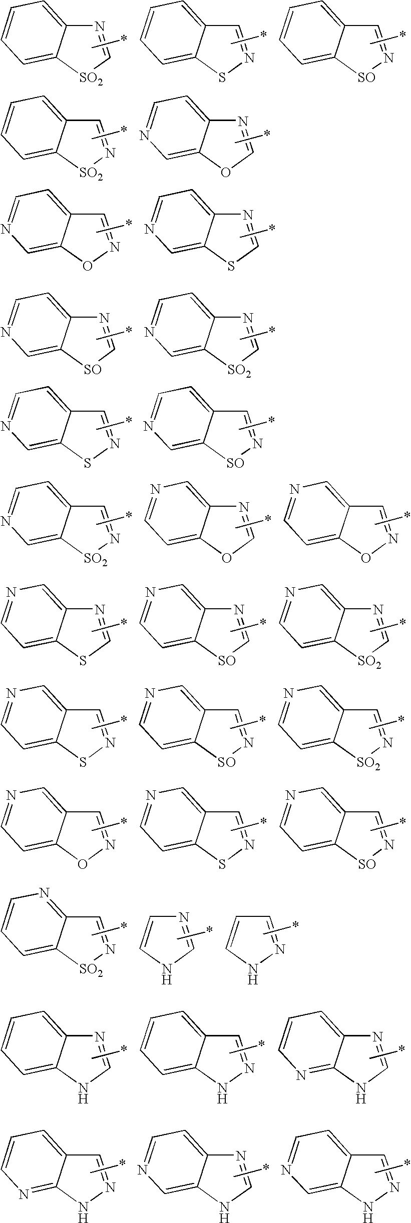

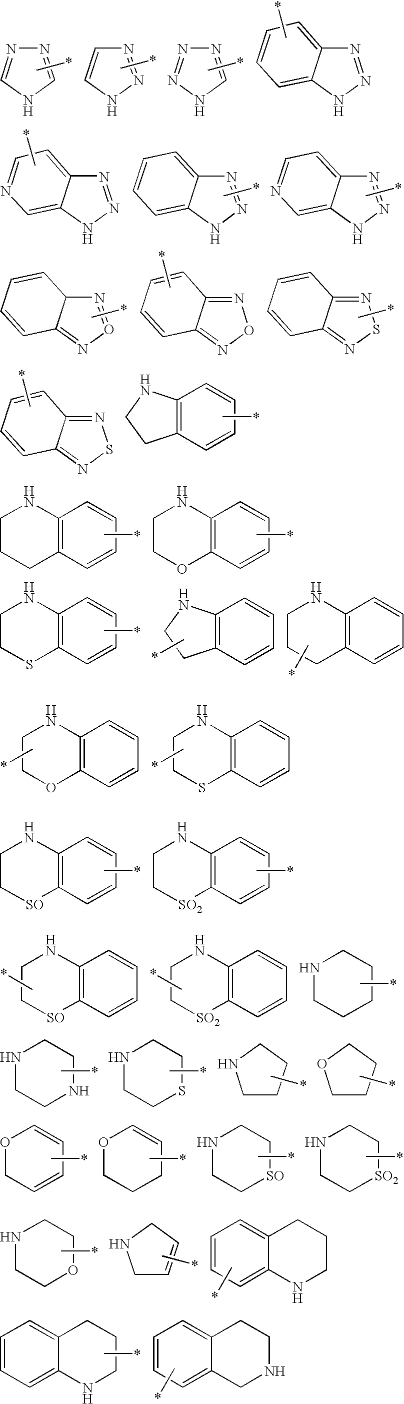

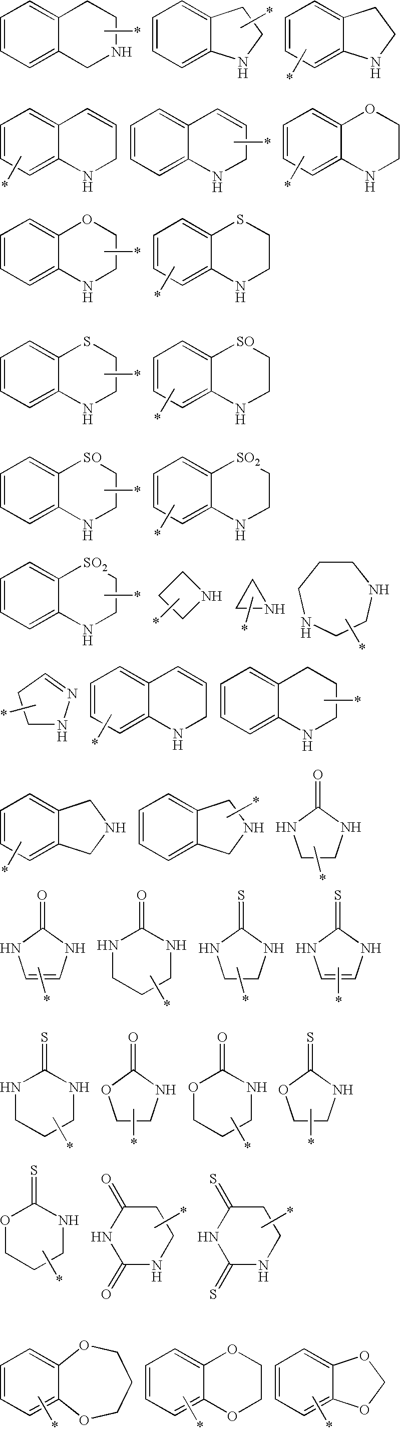

where in each case where an NH is present the bond with the asterisk connecting the respective heterocyclyl moiety to the rest of the molecule the H may be replaced with said bond and/or the H may be replaced by a substituent,

Whenever an unsubstituted or substituted heterocyclyl moiety is present as part of R¹ and R² or is such a substitutent, this heterocyclyl is mono- or bicyclic, that is, it does not have more than two annelated rings (while more rings bound via single bonds which are not annelated, such as aryl substituents or the like, are possible).

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkinyl), and is unsubstituted or substituted by one or more, e.g. one to three substitutents preferably independently selected from those mentioned above as substituents for aryl.

In unsubstituted or substituted aryl-alkyl, aryl (which is preferably unsubstituted or substituted by one or more substituents, e.g. one to three substituents independently selected from those mentioned above as substituents for aryl) is preferably as described above for aryl and is bound to alkyl, preferably C₁-C₇-alkyl, either terminally or at any other carbon in the alkyl chain, e.g. at the 1-carbon.

In unsubstituted or substituted heterocyclyl-alkyl, heterocyclyl is preferably as described above and is unsubstituted or substituted by one or more, e.g. up to three, substitutents independently selected from those mentioned above for substituted aryl, and heterocyclyl is bound to alkyl, preferably C₁-C₇-alkyl, either terminally or at any other carbon in the alkyl chain, e.g. at the 1-carbon.

In unsubstituted or substituted cycloalkyl-alkyl, cycloalkyl is preferably as described above and is unsubstituted or substituted by one or more, e.g. up to three, substitutents independently selected from those mentioned above for substituted aryl, and cycloalkyl is bound to alkyl, preferably C₁-C₇-alkyl, either terminally or at any other carbon in the alkyl chain, e.g. at the 1-carbon.

Unsubstituted or substituted alkyl is preferably C₁-C₂₀-alkyl, more preferably C₁-C₇-alkyl, that is straight-chained or branched (one or, where appropriate, more times), which is unsubstituted or substituted by one or more, e.g. up to three moieties selected from unsubstituted or substituted aryl as described above, especially phenyl or naphthyl each of which is unsubstituted or substituted as described above for unsubstituted or substituted aryl, unsubstituted or substituted heterocycyclyl as described above, especially pyrrolyl, furanyl, thienyl, pyrimidine-2,4-dione-1-, -2-, -3- or -5-yl or benzo[1,3]dioxolyl, which heterocyclyl is unsubstituted or substituted as described above for unsubstituted or substituted heterocyclyl; unsubstituted or substituted cycloalkyl as described above, especially cyclopropyl, cyclobutyl, cyclopentyl or cyclohexyl each of which is unsubstituted or substituted as described above for unsubstituted or substituted cycloalkyl; C₂-C₇-alkenyl, C₂-C₇-alkinyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, hydroxy-C₁-C₇-alkylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, hydroxy-C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkinylene, C₁-C₇-alkylenedioxy, sulfenyl, (—S—OH) sulfonyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl, N-mono or N,N-di-(C₁-C₇-alkyl, phenyl-, naphthyl, phenyl-C₁-C₇-alkyl or naphthyl-C₁-C₇-alkyl)-aminosulfonyl, N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)-aminocarbonylamino and N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)aminosulfonylamino. In cases where unsubstituted or substituted heterocyclyl-alkyl, unsubstituted or substituted aryl-alkyl or unsubstituted or substituted cycloalkyl-alkyl-moieties are mentioned as substituents, the definition of unsubstituted or substituted alkyl relates to such moieties which, in addition to unsubstituted or substituted heterocyclyl, aryl or cycloalkyl comprise at least one further and different moiety (especially from those mentioned in this paragraph) as alkyl substitutent.

Substituted or unsubstituted alkenyl is as defined above for substituted or unsubstituted alkyl, whereby instead of one or more, preferably one, single bond, a double bond is present.

N-mono- or N,N-di-substituted aminocarbonyl is aminocarbonyl (—C(═O)—NH₂) (preferably bound to L=imino or especially oxy) that is mono- or di-substituted at the nitrogen preferably by one or more moieties selected from unsubstituted or substituted alkyl, unsubstituted or substituted aryl, unsubstituted or substituted heterocyclyl or unsubstituted or substituted cycloalkyl, each of which is preferably defined as above; a preferred example is aryl-C₁-C₇-alkylaminocarbonyl (=aryl-C₁-C₇—NH—C(═O)—), such as benzylaminocarbonyl, bound to L=oxy or further imino.

N-mono- or N,N-di-substituted aminosulfonyl is sulfamoyl (—S(═O)₂—NH₂) (preferably bound to L=imino or especially oxy) that is mono- or di-substituted at the nitrogen preferably by one or more moieties selected from unsubstituted or substituted alkyl, unsubstituted or substituted aryl, unsubstituted or substituted heterocyclyl or unsubstituted or substituted cycloalkyl, each of which is preferably defined as above; a preferred example is aryl-C₁-C₇-alkylaminosulfonyl (=aryl-C₁-C₇—NH—S(═O)₂—), such as benzylaminosulfonyl, bound to L=oxy or further imino.

In all definitions above it goes without saying that only stable compounds the person having skill in the art will, without undue experimentation or considerations, be able to recognize are important (e.g. those that are sufficiently stable for the manufacture of pharmaceuticals, e.g. having a half-life of more than 30 seconds) and thus are preferably encompassed by the present claims and that only chemically feasible bonds and substitutions are encompassed, as well as tautomeric forms where present.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

I

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-di-methylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” and “intermediates” hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural or preferably the singular.

The compounds of the present invention possess two or more asymmetric centers depending on the choice of the substituents. The preferred absolute configuration at the C-3 and C-4 asymmetric centers is maintained throughout the specification and the appended claims as indicated herein-above. However, any possible diastereoisomers, enantiomers and geometric isomers, and mixtures thereof, e.g., racemates, are encompassed by the present invention.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that that depends on the activity of beta-secretase and/or the generation of beta-amyloid and the subsequent aggregation into oligomers and fibrils, the use for the manufacture of pharmaceutical compositions for use in the treatment of a disease or disorder that that depends on the activity of beta-secretase and/or the generation of beta-amyloid and the subsequent aggregation into oligomers and fibrils; a method of use of one or more compounds of the formula I in the treatment of a disease or disorder that that depends on the activity of beta-secretase and/or the generation of beta-amyloid and the subsequent aggregation into oligomers and fibrils; a pharmaceutical preparation comprising one or more compounds of the formula I for the treatment of a disease or disorder that that depends on the activity of beta-secretase and/or the generation of beta-amyloid and the subsequent aggregation into oligomers and fibrils; and one or more compounds of the formula I for use in the treatment of a disease or disorder in a warm-blooded animal, especially a human, preferably a disease that that depends on the activity of beta-secretase and/or the generation of beta-amyloid and the subsequent aggregation into oligomers and fibrils; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate.

Highly preferred is a compound of the formula IA with the following configuration:

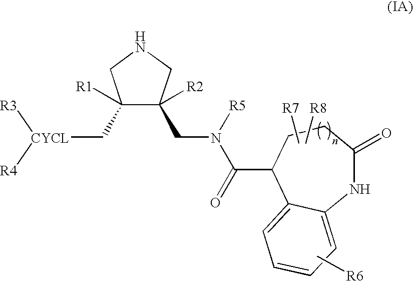

wherein R¹, R², R³, R⁴, R⁵, R⁶, CYCL and n are as defined herein, or a pharmaceutically acceptable salt thereof.

The formula IA can replace formula I wherever a compound of the formula I (including a salt thereof) is mentioned hereinbefore or hereinafter; also, the corresponding intermediates are preferred.

A highly preferred embodiment of the invention relates to a compound of the formula I,

wherein
R¹ is hydrogen;
R² is hydrogen or F;
CYCL is phenyl or cyclohexyl;
R³ and R⁴ are independently of each other hydrogen, C₁-C₇-alkyl, halo-C₁-C₇-alkyl, hydroxy-C₁-C₇-alkyl, halo, hydroxy, or C₁-C₇-alkoxy;
R⁵ is substituted or unsubstituted C₁-C₇-alkyl, C₃-C₇-cycloalkyl, phenyl-C₁-C₇-alkyl, monocyclic heterocyclyl-C₁-C₇-alkyl or C₃-C₇-cycloalkyl-C₁-C₇-alkyl;
n is 0;
R⁶ is hydrogen, C₁-C₇-alkyl, halo-C₁-C₇-alkyl, hydroxy-C₁-C₇-alkyl, halo, hydroxy, or C₁-C₇-alkoxy;
R⁷ is hydrogen, C₁-C₇-alkyl, halo-C₁-C₇-alkyl, hydroxy-C₁-C₇-alkyl, halo, hydroxy, or C₁-C₇-alkoxy;
R⁸ is hydrogen, C₁-C₇-alkyl, halo-C₁-C₇-alkyl, hydroxy-C₁-C₇-alkyl, halo, hydroxy, or C₁-C₇-alkoxy;
or a salt thereof.

### Preferred Definitions for R¹

R¹ is as defined in the claims, preferably R¹ is hydrogen, O-Methyl or halogen, more preferably hydrogen or F, most preferably hydrogen.

### Preferred Definitions for R²

R² is as defined in the claims, preferably R² is hydrogen, O-Methyl or halogen, more preferably hydrogen or F, most preferably hydrogen.

In one embodiment, when R² is F, R¹ is preferably hydrogen.

### Preferred Definitions for CYCL

CYCL is as defined in the claims, preferably, when CYCL is aryl, it is phenyl or naphthyl, more preferably phenyl, or preferably, when CYCL is cycloalkyl, it is C₃-C₇-cycloalkyl, such as cyclopropyl, cyclobutyl, cyclopentyl or cyclohexyl, more preferably cyclohexyl. Most preferably CYCL is phenyl or cyclohexyl.

### Preferred Definitions for R³

R³ is as defined in the claims, preferably R³ is hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, more preferably hydrogen, C₁-C₇-alkyl, halo-C₁-C₇-alkyl, hydroxy-C₁-C₇-alkyl, halo, hydroxy, or C₁-C₇-alkoxy; most preferably hydrogen or halo such as F or Br.

Preferred definitions for R⁴

R⁴ is as defined in the claims, preferably R⁴ is hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, more preferably hydrogen, C₁-C₇-alkyl, halo-C₁-C₇-alkyl, hydroxy-C₁-C₇-alkyl, halo, hydroxy, or C₁-C₇-alkoxy; most preferably hydrogen or halo such as F or Br.

Most preferably, both R³ and R⁴ are hydrogen or a substituent as listed above other than hydrogen, such as halo, or one is hydrogen and the other is a substituent as listed above other than hydrogen, such as halo.

### Preferred Definitions for R⁵

R⁵ is as defined in the claims, preferably R⁵ is unsubstituted or substituted alkyl, unsubstituted or substituted cycloalkyl, unsubstituted or substituted aryl-alkyl, substituted or unsubstituted alkenyl, unsubstituted or substituted mono- or bicyclic heterocyclyl-alkyl or unsubstituted or substituted cycloalkyl-alkyl, more preferably substituted or unsubstituted C₁-C₇-alkyl, C₃-C₇-cycloalkyl, phenyl-C₁-C₇-alkyl, substituted or unsubstituted C₁-C₇-alkenyl, monocyclic heterocyclyl-C₁-C₇-alkyl or C₃-C₇-cycloalkyl-C₁-C₇-alkyl.

In a first embodiment, R⁵ is unsubstituted or substituted alkyl. Preferred examples for alkyl are branched or straight chain C₁-C₇-alkyl which may be substituted or unsubstituted. In one embodiment, R⁵ is branched alkyl such as isopropyl, isobutyl, sec-butyl or tert-butyl, isopentyl, 1-ethylpropyl, and 1,2-dimethyl-propyl, most preferably isopropyl. Branched alkyl is preferably unsubstituted. In another embodiment R⁵ is straight chain alkyl such as methyl, ethyl, n-propyl, n-butyl or n-pentyl, preferably methyl, ethyl or n-propyl. Straight chain alkyl is preferably substituted or unsubstituted. When the alkyl moiety is substituted, it is preferably mono-, di- or tri-substituted, more preferably mono-substituted. Suitable substituents for the alkyl moiety are as defined herein, preferably O—C₁-C₄-alkyl, halo, hydroxy, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, C₁-C₇-alkyloxy-carbonyl or cyano, most preferably, carboxyl or C₁-C₇-alkyloxy-carbonyl.

Preferably in a second embodiment R⁵ is cycloalkyl. Preferred examples for cycloalkyl are monocyclic rings, preferably C₃-C₇-cycloalkyl, more preferably C₃, C₄, C₅ and C₆-cycloalkyl, such as cyclopropyl or cyclobutyl, most preferably cyclopropyl. The cycloalkyl moiety may be substituted or unsubstituted. When the cycloalkyl moiety is substituted, it is preferably mono-substituted. Suitable substituents for the cycloalkyl moiety are as defined herein, preferably O—C₁-C₄-alkyl, halo, hydroxy, unsubstituted or substituted phenyl, naphthyl, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, and cyano. Most preferably, the cycloalkyl moiety is unsubstituted.

Preferably in a third embodiment R² is unsubstituted or substituted aryl-alkyl, such as phenyl-C₁-C₄-alkyl or naphthyl-C₁-C₄-alkyl, preferably phenyl-C₁-C₄-alkyl, such as benzyl, phenethyl, phenyl-CH₂CH₂ CH₂, phenyl-CH₂CH₂ CH₂CH₂, phenyl-CH(CH₃), naphthyl-CH₂, most preferably benzyl. Most preferably, the aryl moiety is phenyl. When the aryl moiety is substituted, it is preferably mono- or di-substituted. Suitable substituents are as defined herein, preferably —(C₀-C₇-alkylene)-(X)ᵣ—(C₁-C₇-alkylene)-(Y)ₛ—(C₀-C₇-alkylene)-H, wherein r and s are 0 or 1 and Y and X are independently O, NH or —NH—CO—O—, —CO—NH—, NHCO, N(C₁-C₇-alkyl), halo-C₁-C₇-alkyl, halo, hydroxy, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, N(mono or di-CO—C₁-C₇-alkyl or formyl)amino, amino-C₁-C₇-alkyl, carboxyl, and cyano. Preferred examples of —(C₀-C₇-alkylene)-(X)ᵣ—(C₁-C₇-alkylene)-(Y)ₛ—(C₀-C₇-alkylene)-H include —(O or NH)—C₁-C₇-alkyl, —CO—NH₂, —C₁-C₇-alkyl, —NHCO—C₁-C₇-alkyl, —(O or NH)—C₁-C₇-alkylene-(O or NH)—C₁-C₇-alkyl, —(O or NH)—C₁-C₇-alkylene-(O or NH)—H, —C₁-C₇-alkylene-(O or NH)—C₁-C₇-alkylene-(O or NH)—C₁-C₇-alkyl, —C₁-C₇-alkylene-(O, NH—CO—O, NHCO or NH)—C₁-C₇-alkyl, —C₁-C₇-alkylene-(O, NHCO or NH)—H, —C₁-C₇-alkylene-N(C₁-C₇-alkyl)-C₁-C₇-alkyl, —C₁-C₇-alkylene-(O or NH)—C₁-C₇-alkylene-(O or NH)—H or —C₁-C₇-alkylene-NH—CO—O—C₁-C₇-alkyl, most preferably —OMe, —CH₂NH₂, —CONH₂, —CH₂N(Me)₂, —CH₂NHCOMe, —CH₂NHCO—H, —CH₂NHC₂H₄OH, NHCOMe, —OC₂H₄OMe, NHCOMe, or —OC₃H₆OMe. Most preferably the aryl moiety is unsubstituted or is substituted with halo, OMe and/or CN.

Preferably in a fourth embodiment, R⁵ is unsubstituted or substituted alkenyl. Preferred examples for alkenyl are branched or straight chain C₁-C₇-alkenyl which may be substituted or unsubstituted. R⁵ contains preferably one or two, more preferably one double bond.

Preferably, R⁵ is one of the following moieties containing one double bond: ethyl, n-propyl, n-butyl or n-pentyl, isopropyl, isobutyl, sec-butyl, isopentyl, 1-ethylpropyl, and 1,2-dimethyl-propyl, most preferably isobutyl. When the alkenyl moiety is substituted, it is preferably mono-, di- or tri-substituted, more preferably mono-substituted. Suitable substituents for the alkyl moiety are as defined herein, preferably O—C₁-C₄-alkyl, halo, hydroxy, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, C₁-C₇-alkyloxy-carbonyl or cyano, most preferably, carboxyl or C₁-C₇-alkyloxy-carbonyl. Most preferably, the alkenyl moiety is unsubstituted.

Preferably in a fifth embodiment R⁵ is unsubstituted or substituted mono- or bicyclic heterocyclyl-alkyl such as heterocyclyl-C₁-C₄-alkyl in particular heterocyclyl-CH₂, heterocyclyl-CH₂CH₂, or heterocyclyl-CH₂CH₂ CH₂, most preferably heterocyclyl-CH₂. The heterocyclic moiety is preferably monocyclic. Preferred are aromatic ring systems, or in particular if a bicyclic moiety is contemplated, partially saturated ring systems, in particular whereby one of the rings is aromatic and the other is saturated or partially saturated, most preferred are aromatic ring systems. The heterocyclyl moiety has preferably 1, 2 or 3, more preferably 1 or 2 heteroatoms selected from O, N or S, more preferably O or N. Particularly preferred examples include pyrrolyl, furanyl, thienyl, pyridyl, pyrimidine-2,4-dione-1-, -2-, -3- or -5-yl, indolyl, benzimidazolyl, benzopyrazolyl, benzofuranyl, quinolinyl, benzo[1,2,5]oxadiazolyl, and 3,4-dihydro-2H-benzo[1,4]oxazinyl, more preferably pyridyl.

When the heterocyclyl moiety is substituted, it is preferably mono-substituted. Suitable substituents for the heterocyclyl moiety are as defined herein, preferably —(C₀-C₇-alkylene)-(X)ᵣ—(C₁-C₇-alkylene)-(Y)ₛ—(C₀-C₇-alkylene)-H, wherein r and s are 0 or 1 and Y and X are independently O, NH or NH—CO—O—, halo-C₁-C₇-alkyl, halo, hydroxy, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, and cyano. Preferred examples of —(C₀-C₇-alkylene)-(X)ᵣ—(C₁-C₇-alkylene)-(Y)ₛ—(C₀-C₇-alkylene)-H include —(O or NH)—C₁-C₇-alkyl, —C₁-C₇-alkyl, —(O or NH)—C₁-C₇-alkylene-(O or NH)—C₁-C₇-alkyl, —(O or NH)—C₁-C₇-alkylene-(O or NH)—H, —C₁-C₇-alkylene-(O or NH)—C₁-C₇-alkylene-(O or NH)—C₁-C₇-alkyl, —C₁-C₇-alkylene-(O or NH)—C₁-C₇-alkyl, or —C₁-C₇-alkylene-NH—CO—O—C₁-C₇-alkyl, more preferably —OMe, —OC₂H₄OMe, —NH-propyl, methyl, ethyl, —C₂H₄—NH—CO-OMe, —CH₂OC₂H₄OMe, —OC₂H₄OC₂H₄, —OC₃H₆OH, —C₂H₄OMe, —C₃H₆OMe and —NH—C₃H₆OMe, yet more preferably —NH-propyl, —C₂H₄OMe and —C₃H₆OMe. Most preferably the heterocyclyl moiety is unsubstituted.

Preferably in a sixth embodiment R⁵ is cycloalkyl alkyl such as cycloalkyl-C₁₋₄ alkyl-, in particular cycloalkyl-CH₂—. Preferred examples for cycloalkyl are monocyclic rings, preferably C₃-C₇-cycloalkyl, more preferably C₃, C₄, C₅ and C₆-cycloalkyl, such as cyclopropyl or cyclobutyl, most preferably cyclopropyl. The cycloalkyl moiety may be substituted or unsubstituted. When the cycloalkyl moiety is substituted, it is preferably mono-substituted.

Suitable substituents for the cycloalkyl moiety are as defined herein, preferably O—C₁-C₄-alkyl, halo, hydroxy, unsubstituted or substituted phenyl, naphthyl, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, and cyano. Most preferably, the cycloalkyl moiety is unsubstituted.

Particularly preferred are the first and second embodiment.

### Preferred Definitions for n

Integer n is as defined in the claims, preferably 0.

### Preferred Definitions for R⁶

R⁶ is as defined in the claims, preferably R⁶ is hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, more preferably hydrogen, C₁-C₇-alkyl, halo-C₁-C₇-alkyl, hydroxy-C₁-C₇-alkyl, halo, hydroxy, or C₁-C₇-alkoxy; most preferably hydrogen or halo such as F.

Preferred definitions for R⁷

R⁷ is as defined in the claims, preferably R⁷ is hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, more preferably hydrogen, C₁-C₇-alkyl, halo-C₁-C₇-alkyl, hydroxy-C₁-C₇-alkyl, halo, hydroxy, or C₁-C₇-alkoxy; most preferably hydrogen or methyl.

Preferred definitions for R⁸

R⁸ is as defined in the claims, preferably R⁸ is hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, more preferably hydrogen, C₁-C₇-alkyl, halo-C₁-C₇-alkyl, hydroxy-C₁-C₇-alkyl, halo, hydroxy, or C₁-C₇-alkoxy; most preferably hydrogen or methyl.

In one embodiment, when R⁷ and R⁸ are both C₁-C₇-alkyl, they may form a C₃-C₇-cycloalkyl ring such as a cyclopropyl ring.

Preferably, both R⁷ and R⁸ are hydrogen or a substituent as listed above other than hydrogen, such as C₁-C₇-alkyl, most preferably both are hydrogen. If both and R⁸ are a substituent as listed above other than hydrogen, such as C₁-C₇-alkyl, they are preferably bonded to the same carbon.

Any of the definitions (including preferred ones) provided for a specific variant may be read in combination with any of the definitions (including preferred ones) of any other variant as is apparent to the person skilled in the art.

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as their use.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof. Preferably the Schemes outlined in the Examples are adopted and, if necessary, adapted to prepare compounds falling under the scope of the present application. Preferably on of the following general methodologies are employed:

A) reacting an aldehyde of the formula II,

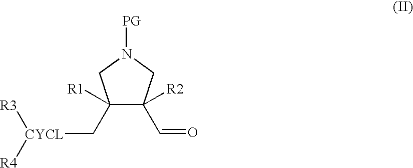

wherein R¹, R², R³, R⁴ and CYCL are as defined for a compound of the formula I and PG is a protecting group, with an amino compound of the formula III

R⁵NH₂ (III)
wherein R⁵ is as defined above under the conditions for reductive amination to obtain a compound of the formula IV wherein R¹, R², R³, R⁴, R⁵ and CYCL are as defined for a compound of the formula I, and then reacting a compound of the formula IV;

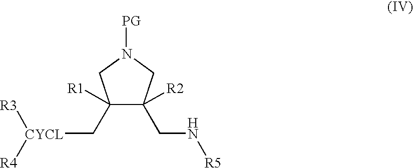

wherein R¹, R², R³, R⁴, R⁵ and CYCL are as defined for a compound of the formula I, and PG is a protecting group, with an acid compound of formula V

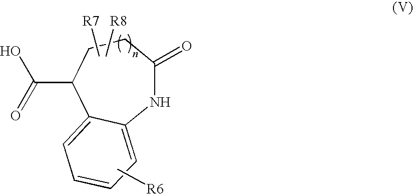

wherein R⁶, R⁷, R⁸ and n are as defined for a compound of the formula I, under condensation conditions to obtain a compound of the formula VI wherein R¹, R², R³, R⁴, R⁵, R⁶, R⁷, R⁸, n and CYCL are as defined for a compound of the formula I, and PG is a protecting group, and then removing the protecting group PG to obtain a compound of the formula I.
   B) reacting an alcohol of the formula VII,

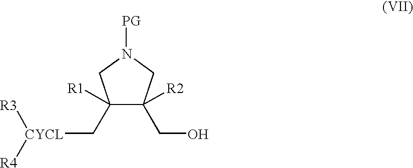

wherein R¹, R², R³, R⁴ and CYCL are as defined for a compound of the formula I and PG is a protecting group, with a sulfonamide of the formula VIII

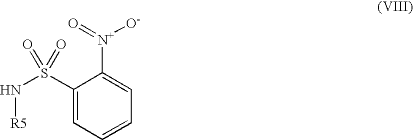

wherein R⁵ is as defined above under conventional Mitsunobu conditions (e.g. Chem. Commun. 2004, 353-359.) to obtain a compound of the formula IX wherein R¹, R², R³, R⁴, R⁵ and CYCL are as defined for a compound of the formula I, and PG is a protecting group, if necessary exchanging the protecting groups, and then reacting a compound of the formula IX;

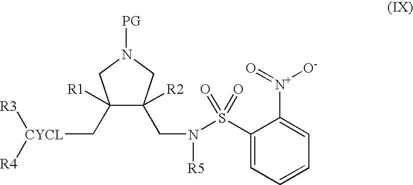

wherein R¹, R², R³, R⁴, R⁵ and CYCL are as defined for a compound of the formula I and PG is a protecting group, under standard deprotection conditions for nitrobenzenesulfonamides (Ns-amides) using thiophenol or thioglycolic acid (e.g. Chem. Commun. 2004, 353-359.) to obtain a compound of the formula IV wherein R¹, R², R³, R⁴, R⁵ and CYCL are as defined for a compound of the formula I, and PG is a protecting group, and then reacting a compound of the formula IV;

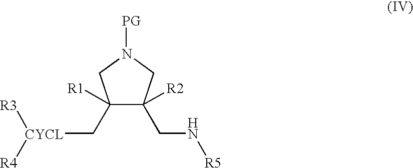

wherein R¹, R², R³, R⁴, R⁵ and CYCL are as defined for a compound of the formula I, and PG is a protecting group, with an acid compound of formula V

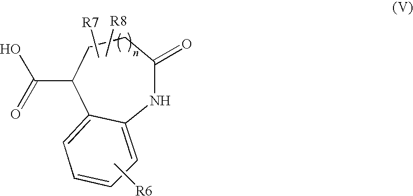

wherein R⁶, R⁷, R⁸ and n are as defined for a compound of the formula I, under condensation conditions to obtain a compound of the formula VI wherein R¹, R², R³, R⁴, R⁵, R⁶, R⁷, R⁸, n and CYCL are as defined for a compound of the formula I, and PG is a protecting group, and then removing the protecting group PG to obtain a compound of the formula I.

If desired, subsequent to any one or more of the processes mentioned under (A) to (B) an obtainable compound of the formula I or a protected form thereof can be converted into a different compound of the formula I, a salt of an obtainable compound of formula I can be converted into the free compound or a different salt, an obtainable free compound of formula I can be converted into a salt thereof, and/or an obtainable mixture of isomers of a compound of formula I can be separated into individual isomers;

where in any of the starting materials, in addition to specific protecting groups mentioned, further protecting groups may be present, and any protecting groups are removed at an appropriate stage in order to obtain the corresponding compound of the formula I, or a salt thereof.

### Preferred Reaction Conditions

The preferred reaction conditions for the reactions mentioned above under A) to B), as well as for the transformations and conversions, are as follows:

The condensation reaction in between an acid of the formula V, or a reactive derivative thereof, and an amino compound of the formula IV preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine or diisopropylethylamine (DIEA) and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP) or 1-(3-dimethylamino-propyl)-3-ethylcarbodiimide hydrochloride/hydroxybenzotriazole (EDCl/HOBT). For review of some other possible coupling agents, see e.g. Klauser; Bodansky, Synthesis 1972, 453-463.

The reaction mixture is preferably stirred at a temperature of between approximately −20 and 50° C., especially between 0° C. and 30° C., e.g. at room temperature. The reaction is preferably carried out under an inert gas, e.g. nitrogen or argon.

The subsequent removal of a protecting group e.g. PG, such as tert-butoxycarbonyl, benzyl or 2-(trimethylsilyl)-ethoxycarbonyl, takes place under standard conditions, see also the literature mentioned below under General Process Conditions. For example, tert-butoxycarbonyl is removed in the presence of an acid, e.g. a TFA or hydrohalic acid, such as HCl, in an appropriate solvent, e.g. an ether, such as dioxane, at customary temperatures, e.g. at room temperature, the removal of benzyl can be achieved e.g. by reaction with ethylchloroformate or 2-trimethylsilylethyl-chloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., and the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetoneitrile, preferably at elevated temperatures, e.g. under reflux conditions.

The reaction between an aldehyde compound of the formula II with an amino compound of the formula III preferably takes place under customary conditions for reductive amination, e.g. in the presence of an appropriate reducing (e.g. hydrogenation) agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature; the subsequent removal of protecting groups takes place e.g. as described above.

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

The reactions can be effected according to conventional methods, for example as described in the Examples.

The working-up of the reaction mixtures and the purification of the compounds thus obtainable may be carried out in accordance with known procedures.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent.

Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods.

Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by chromatography, for example by HPLC, using chromatographic substrates with chiral ligands.

Intermediates and final products can be worked up and/or purified according to customary methods, e.g. using chromatographic methods, distribution methods, (re-) crystallization, and the like.

### Starting Materials

Starting Materials, including intermediates, for compounds of the formula I, such as the compounds of the formulae II, III, V, VII and/or VIII and the like, can be prepared, for example, according to methods that are known in the art, according to methods described in the examples or methods analogous to those described in the examples, and/or they are known or commercially available.

In the subsequent description of starting materials and intermediates and their synthesis, R¹, R², R³, R⁴, R⁵, R⁶, R⁷, R⁸, n and CYCL have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”.

In all formulae above where present, the central pyrrolidine and its substituents at positions 3 and 4 may be present in any one or more of the following configurations, and/or mixtures of the corresponding isomers may be formed and/or separated into the individual isomers at appropriate stages:

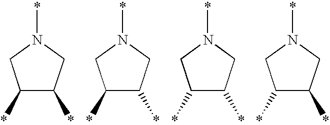

wherein the left lower bond is also on the left side in any of the formulae intermediates or starting materials as shown above or final products of the formula I, the right lower bond on the right side.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/l, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### Pharmaceutical Use, Pharmaceutical Preparations and Methods

Compounds of the formula I and their pharmaceutically acceptable acid addition salts, hereinafter referred to as “agents of the invention”, exhibit valuable pharmacological properties when tested in vitro and in animals, and are therefore useful as medicaments.

The agents of the invention are inhibitors of aspartic proteases and can be used for the treatment of disorders involving processing by such enzymes. Particularly they inhibit beta-secretase and as such inhibit the generation of beta-amyloid and the subsequent aggregation into oligomers and fibrils.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, for the treatment of conditions that depend on the activity of beta-secretase and the generation of beta-amyloid and/or the subsequent aggregation into oligomers and fibrils. Such conditions include Alzheimer's disease, Down's Syndrome, memory and cognitive impairment, dementia, amyloid neuropathies, brain inflammation, nerve and brain trauma, vascular amyloidosis, or cerebral haemorrhage with amyloidosis and the like.

Some of the agents of the invention also inhibit BACE2 (beta-site APP-cleaving enzyme 2) or Cathepsin D, close homologues of the pepsin-type aspartyl proteases and of beta-secretase. Due to the correlation of BACE2 and Cathepsin D expression with a more tumorigenic and metastatic potential of tumor cells, such inhibitors are useful for the suppression of the metastasis process associated with tumor cells.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range from about 10 to about 2000, preferably from about 10 to about 200, mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a medicament, e.g. for the treatment of neurological or vascular disorders related to beta-amyloid generation and/or aggregation.

The present invention furthermore provides a pharmaceutical composition comprising an agent of the invention in association with at least one pharmaceutical carrier or diluent. Such compositions may be manufactured in conventional manner. Unit dosage forms contain, for example, from about 1 to about 1000, preferably from about 1 to about 500, mg of an agent of the invention.

The agents of the invention can be administered alone or in combination with other pharmaceutical agents effective in the treatment of conditions mentioned above.

The pharmaceutical combination may be in the form of a unit dosage form, whereby each unit dosage will comprise a predetermined amount of the two components, in admixture with suitable pharmaceutical carriers or diluents. Alternatively, the combination may be in form of a package containing the two components separately, e.g. a pack or dispenser-device adapted for the concomitant or separate administration of the two active agents, wherein these agents are separately arranged.

Moreover the present invention provides the use of an agent of the invention, for the manufacture of a medicament for the treatment of any neurological or vascular disorders related to beta-amyloid generation and/or aggregation.

In still a further aspect, the present invention provides a method for the treatment of any neurological or vascular disorders related to beta-amyloid generation and/or aggregation, in a subject in need of such treatment, which comprises administering to such subject a therapeutically effective amount of an agent of the invention.

Thus, the pharmacologically active compounds of the invention may be employed in the manufacture of pharmaceutical compositions comprising an effective amount thereof in conjunction or admixture with excipients or carriers suitable for either enteral or parenteral administration. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable sub-stances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Preferably, a compound of the invention is administered to a mammal in need thereof.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

Inter alia the following tests may be used:

### Test 1: Inhibition of Human BACE

Recombinant BACE (extracellular domain, expressed in baculovirus and purified using standard methods) at 0.1-10 nM concentration is incubated with the test compound at various concentrations for 1 hour at room temperature in 10-100 mM acetate buffer, pH 4.5, containing 0.1% CHAPS. Synthetic fluorescence-quenched peptide substrate, derived from the sequence of APP and containing a suitable fluorophore-quencher pair is added to a final concentration of 1-5 μM and the increase in fluorescence is recorded at a suitable excitation/emission wavelength in a microplate spectro-fluorimeter for 5-30 minutes in 1-minute intervals. IC₅₀ values are calculated from percentage of inhibition of BACE-activity as a function of the test compound concentration.

### Test 2: Inhibition of Human BACE-2

Recombinant BACE-2 (extracellular domain, expressed in baculovirus and purified using standard methods) at 0.1-10 nM concentrations is incubated with the test compound at various concentrations for 1 hour at room temperature in 10-100 mM acetate buffer, pH 4.5, containing 0.1% CHAPS. Synthetic peptide substrate derived from the sequence of APP and containing a suitable fluorophore-quencher pair is added to a final concentration of 1-5 μM and the increase in fluorescence is recorded at a suitable excitation/emission wavelength in a microplate spectro-fluorimeter for 5-30 minutes in 1-minute intervals. IC₅₀ values are calculated from percentage of inhibition of BACE-2-activity as a function of the test compound concentration.

### Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in sodium formate or sodium acetate buffer at a suitable pH within the range of pH 3.0-5.0 Synthetic peptide substrate Mca-Gly-Lys-Pro-Ile-Leu-Phe-Phe-Arg-Leu-Lys(DNP)-D-Arg-NH₂ is added to a final concentration of 1-5 μM and the increase in fluorescence is recorded at excitation of 325 nm and emission at 400 nm in a microplate spectro-fluorimeter for 5-30 minutes in 1-minute intervals. IC₅₀ values are calculated from percentage of inhibition of cathepsin D-activity as a function of the test compound concentration.

### Test 4: Inhibition of Cellular Release of Amyloid Peptide 1-40

Chinese hamster ovary cells are transfected with the gene for amyloid precursor protein.

Cells are plated at a density of 8000 cells/well in a 96-well microtiter plate and cultivated for 24 hours in DMEM cell culture medium containing 10% FCS. The test compound is added to the cells at various concentrations, and cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-indicated tests, the agents of the invention show activity at concentrations below 20 μM. specifically, compounds of the formula I, in at least one of the above-indicated tests, preferably show IC₅₀ values in the range from 10 nM to 20 μM.

The following Examples, while representing preferred embodiments of the invention, serve to illustrate the invention without limiting its scope.

### Abbreviations

abs. absolute
Ac acetyl
AcOEt/EtOAc ethyl acetate
AcOH acetic acid
APP amyloid precursor protein
aq aqueous
Ar aryl
Bn benzyl
Bu butyl (nBu=n-butyl, tBu=tert-butyl)
CHAPS 3-[(3-cholamidopropyl)-dimethylammonio]-propansulfonat[
c-hexane cyclohexane
DBU diazabicycloundecen e
DCE 1,2-dichloroethane
DCM dichloromethane
DIAD diisopropyl azodicarboxylate
DIBAL-H diisobutylaluminium hydride
DMEM Dulbecco's modified essential medium
DMF dimethylformamide
DMSO dimethylsulfoxide
DPPA diphenylphosphoryl azide
EDCl 1-(3-dimethylaminopropyl)-3-ethylcarbodiimide hydrochloride
ELISA enzyme-linked immunosorbent assay
Ether diethylether
Et₃N triethylamine
Et₂O diethylether
EtOH ethanol
FCS fetal calf serum
Flow flow rate
h hour(s)
HMPA hexamethylphosphoroamide
HOBt 1-hydroxy benzotriazole
HPLC high performance liquid chromatography
iPrOH isopropanol
L liter(s)
KHMDS potassium hexamethyldisilazane
LC-MS liquid chromatography/mass spectrometry
LDA lithium diisopropylamine
Me methyl
MeI methyl iodide
MeOH methanol
Mg milligram
Min minute(s)
mL milliliter
MS mass spectrometry
NMM 4-methylmorpholine
NMR nuclear magnetic resonance
Pd/C palladium on charcoal
PG protecting group
Ph phenyl
PyBOP (benzotriazol-1-yloxy)-tripyrrolidinophosphonium-hexafluorophosphate
R_{f} ratio of fronts
RMgX benzylmagnesium chloride
RT room temperature
TBAF tetra-butylammonium fluoride
TBDMS-Cl tert-butyldimethylsilyl chloride
TBDMS tert-butyldimethylsilyl
TBME tert-butylmethylether
TEA triethylamine
TEMPO 2,2,6,6,-tetramethyl-1-piperidinyloxy free radical
TFA trifluoroacetic acid
THF tetrahydrofurane
RP reverse phase
RT room temperature
Prep preparative
TLC thin layer chromatography
tr retention time

### Trademarks

Celite=Celite® (The Celite Corporation)=filtering aid based on diatomaceous earth
Nucleosil=Nucleosil®, trademark of Machery & Nagel, Düren, FRG for HPLC materials

Temperatures are measured in degrees Celsius. Unless otherwise indicated, the reactions take place at RT.

TLC conditions: R_{f} values for TLC are measured on 5×10 cm TLC plates, silica gel F₂₅₄, Merck, Darmstadt, Germany.

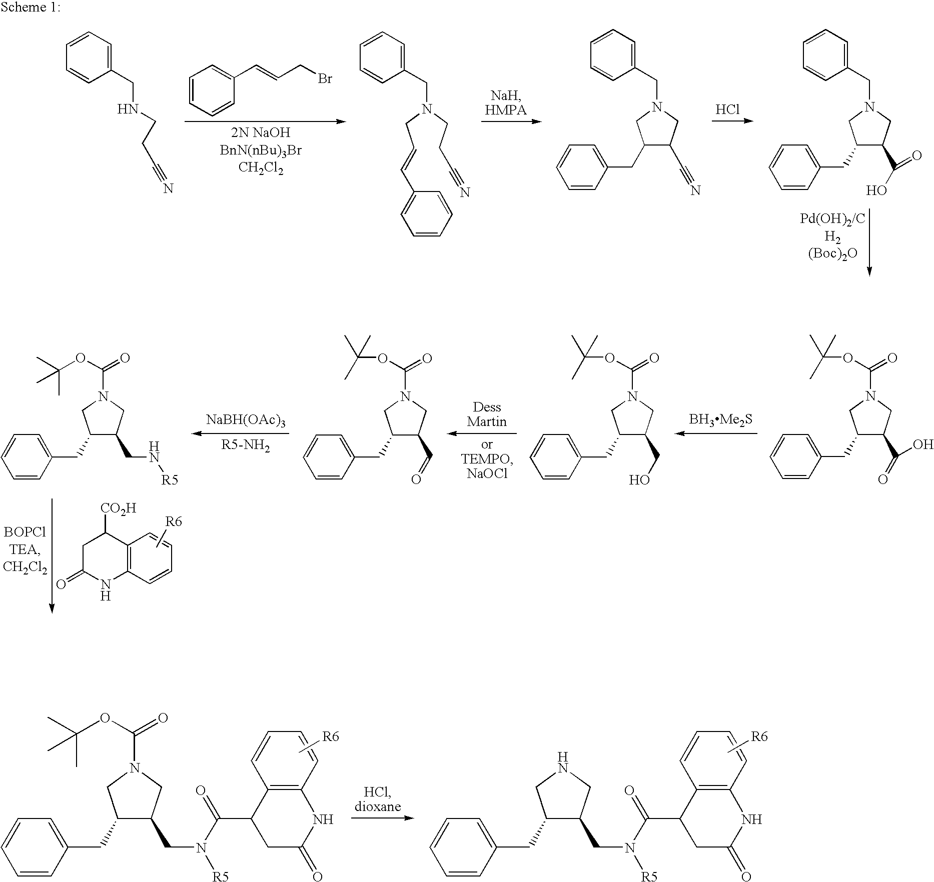

### Example 1

### 2-Oxo-1,2,3,4-tetrahydro-quinoline-4-carboxylic acid ((3S*,4S*)-4-benzyl-pyrrolidin-3-ylmethyl)-methyl-amide

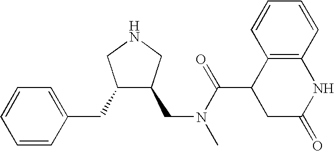

### A. 3-[Benzyl-((E)-3-phenyl-allyl)-amino]-propionitrile

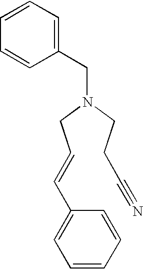

To a solution of 3-benzylamino-propionitrile (4.8 kg, 30 mol) and benzyl-tri-(N-butyl) ammonium bromide (1.08 kg, 3 mol) in 15 L CH₂Cl₂, a 2 N aqueous NaOH solution. (30 L) is added. The reaction mixture is stirred under N₂ atmosphere, and a solution of cinnamyl bromide (5.91 kg, 30 mol) in CH₂Cl₂ (30 L) is added dropwise. The reaction mixture is further stirred at 40° C. for 5 h, diluted with 30 L of CH₂Cl₂ and poured into water (20 L). The layers are separated, and the aqueous one is extracted with CH₂Cl₂. The combined organic layers are dried over MgSO₄, filtered and concentrated under reduced pressure to give the title compound. TLC, Rf (toluene/EtOH, NH₄OH 84/15/1)=0.75.

### B. 1,4-Dibenzyl-pyrrolidine-3-carbonitrile

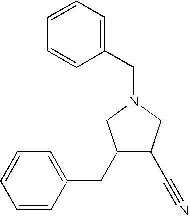

To NaH (80% in grease, 0.816 kg, 27.2 mol), HMPA (17 L) (exothermic!) is carefully added under N₂ atmosphere. The resulting suspension is stirred for 30 min and cooled to 0° C., before dropwise addition of a solution of 3-[benzyl-((E)-3-phenyl-allyl)-amino]-propionitrile (5.98 kg, 18.1 mol) in HMPA (16 L) follows. The reaction mixture is allowed to reach RT overnight and AcOH (1.9 L) is added at 0° C., followed by water (27 L). The reaction mixture is extracted with toluene (3×25 L), the combined organic layers are dried over MgSO₄, filtered and concentrated under reduced pressure to give the title compound as a brown oil. To a solution of the residue in EtOH abs. (5 L), a solution of oxalic acid monohydrate (2.28 kg, 18.1 mol) in EtOH abs. (4 L) is added, and the resulting mixture is stirred at RT. 8 L of Et₂O are added, and the mixture is further stirred for 1 h at 5 C. Acetone and Et₂O 1/1 (6 L) are added, the mixture is centrifuged, and the residue is further washed with Et₂O and filtered. The resulting material is dried under vacuum to give the desired oxalate salt. To a solution of the oxalate salt (3.3 kg) in a mixture of water (20 L) and toluene (33 L), NH₄OH (25%, 2.6 L) is added to adjust the pH to 10. The layers are separated, and the aqueous one is extracted twice with toluene (10 L). The combined organic layers are dried over MgSO₄, filtered and concentrated to give the title compound. TLC, Rf (CH₂Cl₂/benzene 75/25)=0.5.

### C. (3S*,4S*)-1,4-Dibenzyl-pyrrolidine-3-carboxylic acid

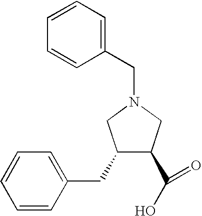

A solution of 1,4-dibenzyl-pyrrolidine-3-carbonitrile (0.828 kg, 3 mol), acetic acid (2.7 L), water (0.9 L) and concentrated HCl (0.9 L) is refluxed for 18 h. To the solution, charcoal is added, and the resulting mixture is further stirred at 50° C. before filtration of the (still warm) mixture on a pad of Celite. The filtrate is concentrated under reduced pressure, and the residue is dissolved into a mixture of MeOH (1.5 L) and water (7.5 L) at 50° C. To the resulting solution, H₂O (7.5 L) and toluene (4.5 L) are added, the layers are separated, and the aqueous one is back-extracted with toluene (4 L). Charcoal is added to the aqueous layer, and, after filtration on a pad of Celite, the filtrate is basified to pH 6 by addition at 60° C. of an aqueous ammonia solution (10%) to allow the ammonium salt to precipitate out. The mixture is vigorously stirred for 45 min and allowed to cool to RT before filtration. The resulting compound is re-crystallized in EtOH to give the title compound. TLC, Rf (CH₂Cl₂/MeOH/NH₄OH 50/45/5)=0.45. MS (LC-MS): 296 [M+H]⁺; t_{R} (HPLC, Nucleosil C18 column, 10-100% CH₃CN/H₂O in 5 min, 100% CH₃CN/3 min, flow: 1.5 mL/min): 4.15 min.

### D. (3S*,4S*)-4-Benzyl-pyrrolidine-1,3-dicarboxylic acid 1-tert-butyl ester

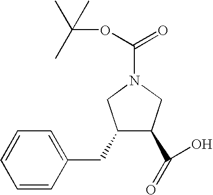

A mixture of (3R*,4R*)-1,4-dibenzyl-pyrrolidine-3-carboxylic acid (50 g, 0.169 mol), di-tert-butylcarbonate (37.1 g, 0.169 mol) and Pd(OH)₂/C 20% (5 g, 50% wet) in EtOH (1 L) is stirred under an hydrogen atmosphere for 6 h. The crude material is filtered over a pad of Celite, dried over Na₂SO₄, filtered and concentrated under reduced pressure to give the title compound. TLC, Rf (CH₂Cl₂/MeOH 95/5)=0.33. MS (LC-MS): 304.2 [M+H]⁺.

### E. (3S*,4S*)-3-Benz yl-4-hydroxymethyl-pyrrolidine-1-carboxylic acid tert-butyl ester

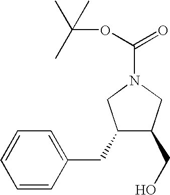

To a solution of (3S*,4S*)-4-benzyl-pyrrolidine-1,3-dicarboxylic acid 1-tert-butyl ester (47.2 g, 0.154 mol) in THF (340 mL), a solution of borane dimethylsulfide complex (2N in THF, 123.5 mL, 0.247 mol) is slowly added at −10° C. The mixture is stirred for 80 min at −10° C. then allowed to reach RT and further stirred overnight. The mixture is carefully poured into MeOH and concentrated under reduced pressure. The residue is taken up in CH₂Cl₂ and extracted with an aqueous saturated solution of NaHCO₃. The organic layer is dried over Na₂SO₄, filtered and concentrated under reduced pressure to afford the title compound. TLC, Rf (CH₂Cl₂/MeOH 90/10)=0.6. t_{R} (HPLC, Nucleosil C18 column, 10 to 90% CH₃CN in H₂O in 11 min, CH₃CN and H₂O containing 0.1% TFA, flow: 1.5 mL/min): 5.29 min.

### (3R,4R)-3-Benzyl-4-hydroxymethyl-pyrrolidine-1-carboxylic acid tert-butyl ester and (3S,4S)-3-Benz yl-4-hydroxymethyl-pyrrolidine-1-carboxylic acid tert-butyl ester

The two enantiomers are separated via chiral preparative HPLC (Chiracel OJ, Daicel Chemical Industries, LTD.) 10×50 cm 20 um, flow: 120 mL/min, UV=210 nM, injection=1.2 g) (eluent: heptane/EtOH 85/45):

(3R,4R)-3-Benzyl-4-hydroxymethyl-pyrrolidine-1-carboxylic acid tert-butyl ester: t_{R} 32.5 min.

(3S,4S)-3-Benzyl-4-hydroxymethyl-pyrrolidine-1-carboxylic acid tert-butyl ester: t_{R} 40.9 min.

### F. Alternative a) (3S*,4S*)-3-Benzyl-4-formyl-pyrrolidine-1-carboxylic acid tert-butyl ester

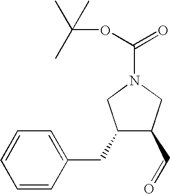

To a well-stirred mixture of (3S*,4S*)-3-benzyl-4-hydroxymethyl-pyrrolidine-1-carboxylic acid tert-butyl ester (10 g, 34.3 mol) and Dess-Martin periodinane (14.55 g, 34.3 mmol) in CH₂Cl₂ (200 mL), slowly wet CH₂Cl₂ (37.7 mmol, 0.68 mL of water in 50 mL of CH₂Cl₂) is added. The clear solution becomes cloudy towards the end of wet CH₂Cl₂ addition. The mixture is diluted with Et₂O and concentrated to a few mL of solvent by rotary evaporation. The residue is taken up in Et₂O, and washed with a 1/1 10% Na₂S₂O₃/saturated aqueous solution of NaHCO₃, followed by H₂O and brine. The aqueous washings are back-extracted with Et₂O, and this organic layer is washed with H₂O and brine. The combined organic layers are dried over Na₂SO₄, filtered and concentrated under reduced pressure. The crude material is purified by flash chromatography on silica gel (eluent; c-hexane/AcOEt 2/1) to give the title compound as a slightly yellow oil. TLC, Rf (c-hexane/AcOEt 2/1)=0.5. ¹H NMR (DMSO, 400 MHz): δ=1.49 (s, 9H), 2.7-2.88 (m, 4H), 3.08 (dd, 1H), 3.32 (dd, 1H), 3.48-3.58 (m, 2H), 7.23 (m, 3H), 7.32 (m, 2H), 9.5 (m, 1H).

### Alternative b) (3S*,4S*)-3-Benzyl-4-formyl-pyrrolidine-1-carboxylic acid tert-butyl ester

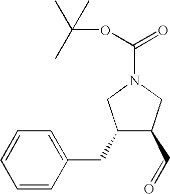

To a mixture of (3S*,4S*)-3-benzyl-4-hydroxymethyl-pyrrolidine-1-carboxylic acid tert-butyl ester (60 g, 0.206 mol) and TEMPO (0.96 g, 0.006 mol) in toluene/AcOEt (1/1, 2 L), at RT a solution of KBr (36.6 g, 0.309 mol) in water (100 mL) is added. The resulting mixture is cooled to 0° C., before the dropwise addition of a water (1 L) solution containing KHCO₃ (77.4 g, 0.773 mol) and NaOCl (57.5 g, 0.772 mol) follows. The resulting reaction mixture is further stirred for 1 h at 0° C. and 3 h at RT. The layers are separated, and the aqueous one is back-extracted twice with toluene/AcOEt (1/1, 500 mL). The combined organic extracts are washed with a solution (3 L) containing water/10% aqueous solution of Na₂S₂O₃/10% aqueous solution of KHSO₄ (1/1/1), dried over Na₂SO₄, filtered and concentrated under reduced pressure. The crude material is purified by flash chromatography on silica gel (eluent; c-hexane/AcOEt 2/1) to give the title compound as a slightly yellow oil.

### G. (3S*,4R*)-3-Benzyl-4-methylaminomethyl-pyrrolidine-1-carboxylic acid tert-butyl ester

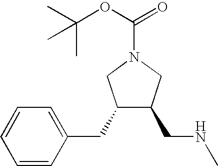

(3S*,4S*)-3-Benzyl-4-formyl-pyrrolidine-1-carboxylic acid tert-butyl ester (250 mg, 0.86 mmol) and methylamine (0.12 mL, 0.95 mmol, 33% in EtOH) are mixed in 1,2-dichloroethane (20 mL) and treated with sodium triacetoxyborohydride (0.31 g, 1.30 mmol) at 0° C. The mixture is stirred at RT under nitrogen overnight, quenched by addition of aqueous saturated NaHCO₃-solution, extracted with CH₂Cl₂, dried over Na₂SO₄ and concentrated. The crude product is purified by präp. RP-HPLC (eluent: ACN/H₂O) to give the title compound. TLC, Rf (CH₂Cl₂/MeOH 9/1)=0.28

MS (EI+): 305 [M+H]⁺

### H. (3S*,4R*)-3-Benzyl-4-{[methyl-(2-oxo-1,2,3,4-tetrahydro-quinoline-4-carbonyl)-amino]-methyl}-pyrrolidine-1-carboxylic acid tert-butyl ester

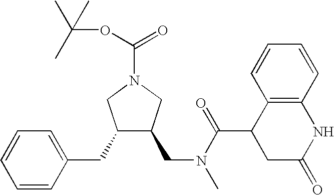

A mixture of (3S*,4R*)-3-Benzyl-4-methylaminomethyl-pyrrolidine-1-carboxylic acid tert-butyl ester (126 mg, 0.41 mmol), BOP-Cl (163 mg, 0.62 mmol), NEt₃ (0.29 mL, 2.1 mmol) and 2-Oxo-1,2,3,4-tetrahydro-quinoline-4-carboxylic acid (87 mg, 0.46 mmol) in CH₂Cl₂ (10 mL) is refluxed for 2.5 h. The reaction mixture is quenched with water extracted with CH₂Cl₂, dried with Na₂SO₄, filtered and the solvent is removed in vacuo. The crude product is purified by RP präp. HPLC

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.65 min. TLC, Rf (CH₂Cl₂/MeOH/H₂O/AcOH 90/10/1/0.5)=0.09, MS (EI+): 478 [M+H]⁺

### I. 2-Oxo-1,2,3,4-tetrahydro-quinoline-4-carboxylic acid ((3S*,4S*)-4-benzyl-pyrrolidin-3-ylmethyl)-methyl-amide

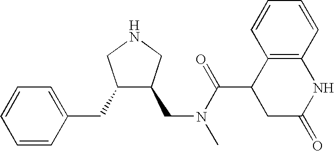

To a solution of (3S*,4R*)-3-Benzyl-4-{[methyl-(2-oxo-1,2,3,4-tetrahydro-quinoline-4-carbonyl)-amino]-methyl}-pyrrolidine-1-carboxylic acid tert-butyl ester (0.169 g, 0.35 mmol) in 1,4-dioxane (4 mL), 4N HCl (5 mL) in 1,4-dioxane is added. After stirring for 5 h, the solvent is removed in vacuo, and the residue is lyophilized overnight to give the title compound as a hydrochloride salt.

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 2.70 min. TLC, Rf (CH₂Cl₂/MeOH/H₂O/AcOH 90/10/1/0.5)=0.57. MS (EI+): 378 [M+H]⁺

### Example 2

### 2-Oxo-1,2,3,4-tetrahydro-quinoline-4-carboxylic acid ((3S*,4S*)-4-benzyl-pyrrolidin-3-ylmethyl)-cyclopropyl-amide

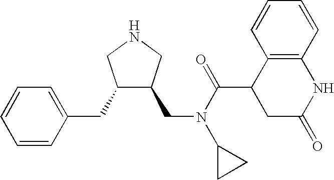

### A. (3S*,4R*)-3-Benzyl-4-cyclopropylaminomethyl-pyrrolidine-1-carboxylic acid tert-butyl ester

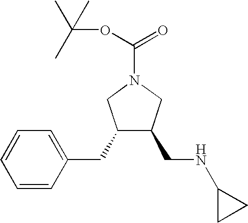

The title compound is prepared analogously as described for the title compound under G in Example 1 (Scheme 1) using (3S*,4S*)-3-Benzyl-4-formyl-pyrrolidine-1-carboxylic acid tert-butyl ester (500 mg, 1.73 mmol) and cyclopropylamine (131 mg, 2.5 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.91 min. TLC, Rf (CH₂Cl₂/MeOH 9/1)=0.63, MS (EI+): 331 [M+H]⁺

### B. (3S*,4R*)-3-Benzyl-4-{[cyclopropyl-(2-oxo-1,2,3,4-tetrahydro-quinoline-4-carbonyl)-amino]-methyl}-pyrrolidine-1-carboxylic acid tert-butyl ester

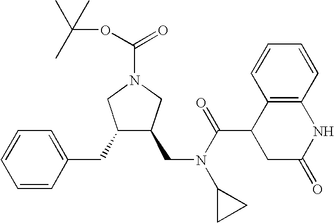

The title compound is prepared analogously as described for the title compound under H in Example 1 (Scheme 1) using (3S*,4R*)-3-Benzyl-4-cyclopropylaminomethyl-pyrrolidine-1-carboxylic acid tert-butyl ester (358 mg, 1.1 mmol) and 2-Oxo-1,2,3,4-tetrahydro-quinoline-4-carboxylic acid (269 mg, 1.4 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.98 min. TLC, Rf (CH₂Cl₂/MeOH 19/1)=0.19, MS (EI+): 504 [M+H]⁺

### C. 2-Oxo-1,2,3,4-tetrahydro-quinoline-4-carboxylic acid ((3S*,4S*)-4-benzyl-pyrrolidin-3-ylmethyl)-cyclopropyl-amide

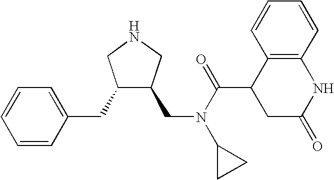

The title compound is prepared analogously as described for the title compound under I in Example 1 (Scheme 1) using (3S*,4R*)-3-Benzyl-4-{[cyclopropyl-(2-oxo-1,2,3,4-tetrahydro-quinoline-4-carbonyl)-amino]-methyl}-pyrrolidine-1-carboxylic acid tert-butyl ester (543 mg, 1.1 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.15 min. TLC, Rf (CH₂Cl₂/MeOH/H₂O/AcOH 90/10/1/0.5)=0.15. MS (EI+): 404 [M+H]⁺

### Example 3

### 2-Oxo-1,2,3,4-tetrahydro-quinoline-4-carboxylic acid benzyl-((3S*,4S*)-4-benzyl-pyrrolidin-3-ylmethyl)-amide

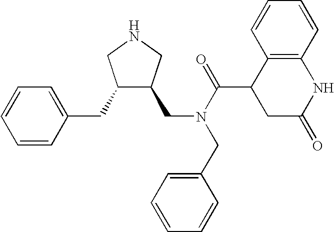

### A. (3S*,4R*)-3-Benzyl-4-(benzylamino-methyl)-pyrrolidine-1-carboxylic acid tert-butyl ester

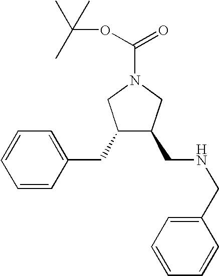

The title compound is prepared analogously as described for the title compound under G in Example 1 (Scheme 1) using (3S*,4S*)-3-Benzyl-4-formyl-pyrrolidine-1-carboxylic acid tert-butyl ester (300 mg, 1.0 mmol) and benzylamine (134 mg, 1.3 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.22 min. TLC, Rf (CH₂Cl₂/MeOH 98/2)=0.12, MS (EI+): 381 [M+H]⁺

### B. (3S*,4R*)-3-Benzyl-4-{[benzyl-(2-oxo-1,2,3,4-tetrahydro-quinoline-4-carbonyl)-amino]-methyl}-pyrrolidine-1-carboxylic acid tert-butyl ester

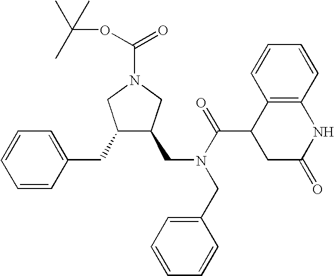

The title compound is prepared analogously as described for the title compound under H in Example 1 (Scheme 1) using (3S*,4R*)-3-Benzyl-4-(benzylamino-methyl)-pyrrolidine-1-carboxylic acid tert-butyl ester (597 mg, 1.6 mmol) and 2-Oxo-1,2,3,4-tetrahydro-quinoline-4-carboxylic acid (390 mg, 2.0 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.33 min. TLC, Rf (CH₂Cl₂/MeOH 19/1)=0.17, MS (EI+): 554 [M+H]⁺

### C. 2-Oxo-1,2,3,4-tetrahydro-quinoline-4-carboxylic acid benzyl-((3S*,4S*)-4-benzyl-pyrrolidin-3-ylmethyl)-amide

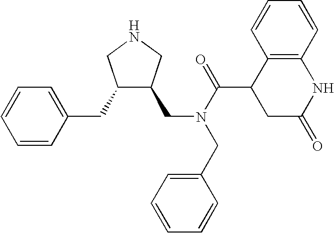

The title compound is prepared analogously as described for the title compound under I in Example 1 (Scheme 1) using (3S*,4R*)-3-Benzyl-4-{[benzyl-(2-oxo-,2,3,4-tetrahydro-quinoline-4-carbonyl)-amino]-methyl}-pyrrolidine-1-carboxylic acid tert-butyl ester (684 mg, 1.1 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.75 min. TLC, Rf (CH₂Cl₂/MeOH/H₂O/AcOH 90/10/1/0.5)=0.15. MS (EI+): 454 [M+H]⁺

### Example 4

### 2-Oxo-1,2,3,4-tetrahydro-quinoline-4-carboxylic acid ((3S*,4S*)-4-benzyl-pyrrolidin-3-ylmethyl)-cyclopropylmethyl-amide

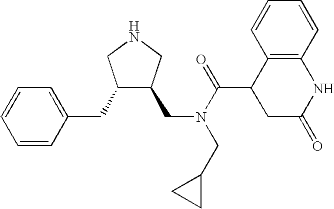

### A. (3S*,4R*)-3-Benzyl-4-[(cyclopropylmethyl-amino)-methyl]-pyrrolidine-1-carboxylic acid tert-butyl ester

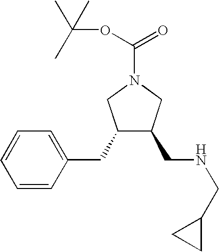

The title compound is prepared analogously as described for the title compound under G in Example 1 (Scheme 1) using (3S*,4S*)-3-Benzyl-4-formyl-pyrrolidine-1-carboxylic acid tert-butyl ester (500 mg, 1.73 mmol) and C-Cyclopropyl methylamine (166 mg, 2.25 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.10 min. TLC, Rf (CH₂Cl₂/MeOH 9/1)=0.39, MS (EI+): 345 [M+H]⁺

### B. (3S*,4R*)-3-Benzyl-4-{[cyclopropylmethyl-(2-oxo-1,2,3,4-tetrahydro-quinoline-4-carbonyl)-amino]-methyl}-pyrrolidine-1-carboxylic acid tert-butyl ester

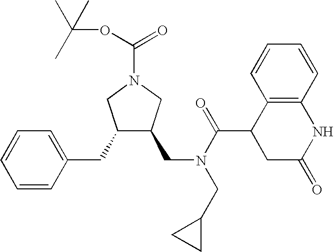

The title compound is prepared analogously as described for the title compound under H in Example 1 (Scheme 1) using (3S*,4R*)-3-Benzyl-4-[(cyclopropylmethyl-amino)-methyl]-pyrrolidine-1-carboxylic acid tert-butyl ester (491 mg, 1.4 mmol) and 2-Oxo-1,2,3,4-tetrahydro-quinoline-4-carboxylic acid (354 mg, 1.8 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.12 min. TLC, Rf (CH₂Cl₂/MeOH 19/1)=0.17, MS (EI+): 518 [M+H]⁺

### C. 2-Oxo-1,2,3,4-tetrahydro-quinoline-4-carboxylic acid ((3S*,4S*)-4-benzyl-pyrrolidin-3-ylmethyl)-cyclopropylmethyl-amide

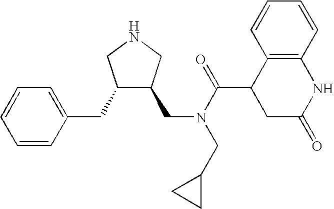

The title compound is prepared analogously as described for the title compound under I in Example 1 (Scheme 1) using (3S*,4R*)-3-Benzyl-4-{[cyclopropylmethyl-(2-oxo-1,2,3,4-tetrahydro-quinoline-4-carbonyl)-amino]-methyl}-pyrrolidine-1-carboxylic acid tert-butyl ester (618 mg, 1.2 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.45 min. TLC, Rf (CH₂Cl₂/MeOH/H₂O/AcOH 90/10/1/0.5)=0.14. MS (EI+): 418 [M+H]⁺

### Example 5

### 2-Oxo-1,2,3,4-tetrahydro-quinoline-4-carboxylic acid ((3S*,4S*)-4-benzyl-pyrrolidin-3-ylmethyl)-(2-methyl-allyl)-amide

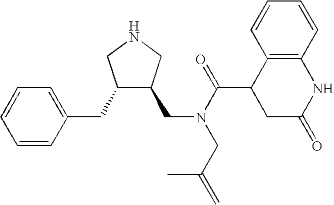

### A. (3S*,4R*)-3-Benzyl-4-[(2-methyl-allylamino)-methyl]-pyrrolidine-1-carboxylic acid tert-butyl ester

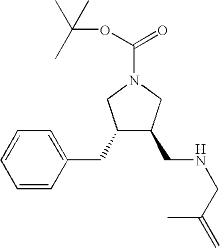

The title compound is prepared analogously as described for the title compound under G in Example 1 (Scheme 1) using (3S*,4S*)-3-Benzyl-4-formyl-pyrrolidine-1-carboxylic acid tert-butyl ester (500 mg, 1.73 mmol) and 2-Methylallylamine (165 mg, 2.25 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.13 min. TLC, Rf (CH₂Cl₂/MeOH 9/1)=0.59, MS (EI+): 345 [M+H]⁺

### B. (3S*,4R*)-3-Benz yl-4-[(2-methyl-allyl)-(2-oxo-1,2,3,4-tetrahydro-quinoline-4-carbonyl)-amino]-methyl)-pyrrolidine-1-carboxylic acid tert-butyl ester

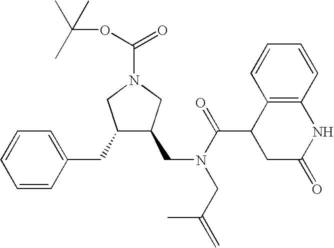

The title compound is prepared analogously as described for the title compound under H in Example 1 (Scheme 1) using (3S*,4R*)-3-Benzyl-4-[(2-methyl-allylamino)-methyl]-pyrrolidine-1-carboxylic acid tert-butyl ester (272 mg, 0.79 mmol) and 2-Oxo-1,2,3,4-tetrahydro-quinoline-4-carboxylic acid (196 mg, 1.3 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.18 min. TLC, Rf (CH₂Cl₂/MeOH 19/1)=0.19, MS (EI+): 518 [M+H]⁺

### C. 2-Oxo-1,2,3,4-tetrahydro-quinoline-4-carboxylic acid ((3S*,4S*)-4-benzyl-pyrrolidin-3-ylmethyl)-(2-methyl-allyl)-amide

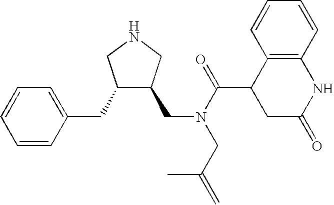

The title compound is prepared analogously as described for the title compound under I in Example 1 (Scheme 1) using (3S*,4R*)-3-Benzyl-4-{[(2-methyl-allyl)-(2-oxo-1,2,3,4-tetrahydro-quinoline-4-carbonyl)-amino]-methyl}-pyrrolidine-1-carboxylic acid tert-butyl ester (336 mg, 0.65 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 2.76 min. TLC, Rf (CH₂Cl₂/MeOH/H₂O/AcOH 90/10/1/0.5)=0.08. MS (EI+): 436 [M+18]⁺

### Example 6

### 2-Oxo-1,2,3,4-tetrahydro-quinoline-4-carboxylic acid ((3S*,4S*)-4-benzyl-pyrrolidin-3-ylmethyl)-isopropyl-amide

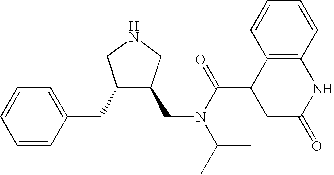

### A. (3S*,4R*)-3-Benzyl-4-isopropylaminomethyl-pyrrolidine-1-carboxylic acid tert-butyl ester

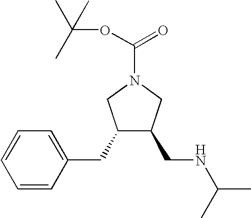

The title compound is prepared analogously as described for the title compound under G in Example 1 (Scheme 1) using (3S*,4S*)-3-Benzyl-4-formyl-pyrrolidine-1-carboxylic acid tert-butyl ester (220 mg, 0.76 mmol) and isopropylamine (100 mL, 1.14 mmol). MS (EI+): 333.3 [M+H]⁺

### B. (3S*,4R*)-3-Benzyl-4-{[isopropyl-(2-oxo-1,2,3,4-tetrahydro-quinoline-4-carbonyl)-amino]-methyl}-pyrrolidine-1-carboxylic acid tert-butyl ester

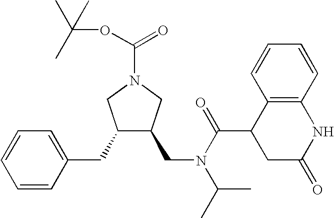

The title compound is prepared analogously as described for the title compound under H in Example 1 (Scheme 1) using (3S*,4R*)-3-Benzyl-4-isopropylaminomethyl-pyrrolidine-1-carboxylic acid tert-butyl ester (200 mg, 0.6 mmol) and 2-Oxo-1,2,3,4-tetrahydro-quinoline-4-carboxylic acid (173 mg, 0.9 mmol). MS (EI+): 506.3 [M+H]⁺

### C. 2-Oxo-1,2,3,4-tetrahydro-quinoline-4-carboxylic acid ((3S*,4S*)-4-benzyl-pyrrolidin-3-ylmethyl)-isopropyl-amide

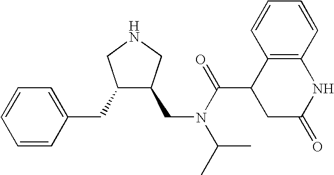

The title compound is prepared analogously as described for the title compound under I in Example 1 (Scheme 1) using (3S*,4R*)-3-Benzyl-4-{[isopropyl-(2-oxo-1,2,3,4-tetrahydro-quinoline-4-carbonyl)-amino]-methyl}-pyrrolidine-1-carboxylic acid tert-butyl ester (200 mg, 0.4 mmol). MS (EI+): 406 [M+H]⁺. t_{R} (HPLC, Nucleosil C18 column, 10-100% CH₃CN/H₂O 5 min, 100% CH₃CN/2.5 min, CH₃CN and H₂O containing 0.1% TFA, flow: 1.0 mL/min): 4.28 min.

### Example 7

### 2-Oxo-1,2,3,4-tetrahydro-quinoline-4-carboxylic acid ((3S*,4S*)-4-benzyl-pyrrolidin-3-ylmethyl)-cyclobutyl-amide

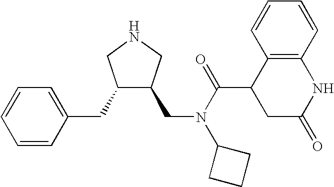

### A. (3S*,4R*)-3-Benzyl-4-cyclobutylaminomethyl-pyrrolidine-1-carboxylic acid tert-butyl ester

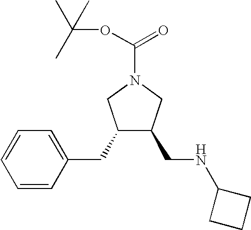

The title compound is prepared analogously as described for the title compound under G in Example 1 (Scheme 1) using (3S*,4S*)-3-Benzyl-4-formyl-pyrrolidine-1-carboxylic acid tert-butyl ester (300 mg, 1.04 mmol) and cyclobutylamine (111 mg, 1.56 mmol). MS (EI+): 345.2 [M+H]⁺

### B. (3S*,4R*)-3-Benzyl-4-{[cyclobutyl-(2-oxo-1,2,3,4-tetrahydro-quinoline-4-carbonyl)-amino]-methyl}-pyrrolidine-1-carboxylic acid tert-butyl ester

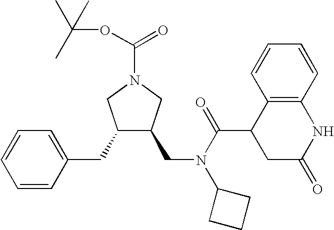

The title compound is prepared analogously as described for the title compound under H in Example 1 (Scheme 1) using (3S*4R*)-3-Benzyl-4-cyclobutylaminomethyl-pyrrolidine-1-carboxylic acid tert-butyl ester (140 mg, 0.4 mmol) and 2-Oxo-1,2,3,4-tetrahydro-quinoline-4-carboxylic acid (93 mg, 0.48 mmol). MS (EI+): 518.3 [M+H]⁺

### C. 2-Oxo-1,2,3,4-tetrahydro-quinoline-4-carboxylic acid ((3S*,4S*)-4-benzyl-pyrrolidin-3-ylmethyl)-cyclobutyl-amide

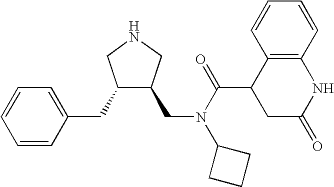

The title compound is prepared analogously as described for the title compound under I in Example 1 (Scheme 1) using (3S*,4R*)-3-Benzyl-4-{[isopropyl-(2-oxo-1,2,3,4-tetrahydro-quinoline-4-carbonyl)-amino]-methyl}-pyrrolidine-1-carboxylic acid tert-butyl ester (200 mg, 0.38 mmol). MS (EI+): 455 [M+H]⁺. t_{R} (HPLC, Nucleosil C18 column, 10-100% CH₃CN/H₂O 5 min, 100% CH₃CN/2.5 min, CH₃CN and H₂O containing 0.1% TFA, flow: 1.0 mL/min): 4.98 and 5.06 min.

### Example 8

### 6-Fluoro-2-oxo-1,2,3,4-tetrahydro-quinoline-4-carboxylic acid ((3S*,4S*)-4-benzyl-pyrrolidin-3-ylmethyl)-cyclopropyl-amide

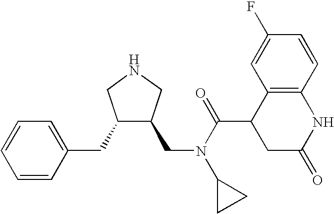

### A. (3S*,4R*)-3-Benzyl-4-{[cyclopropyl-(6-fluoro-2-oxo-1,2,3,4-tetrahydro-quinoline-4-carbonyl)-amino]-methyl}-pyrrolidine-1-carboxylic acid tert-butyl ester

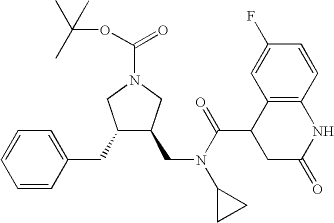

The title compound is prepared analogously as described for the title compound under B in Example 2 (Scheme 1) using (3S*,4R*)-3-Benzyl-4-cyclopropylaminomethyl-pyrrolidine-1-carboxylic acid tert-butyl ester (100 mg, 0.3 mmol) and 6-Fluoro-2-oxo-1,2,3,4-tetrahydro-quinoline-4-carboxylic acid (188 mg, 0.9 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.03 min. TLC, Rf (CH₂C₂/MeOH 19/1)=0.31, MS (EI+): 522 [M+H]⁺

### B. 6-Fluoro-2-oxo-1,2,3,4-tetrahydro-quinoline-4-carboxylic acid ((3S*4S*)-4-benzyl-pyrrolidin-3-ylmethyl)-cyclopropyl-amide

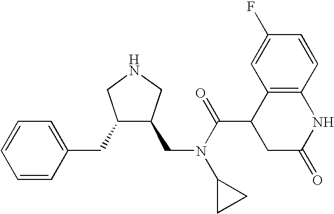

The title compound is prepared analogously as described for the title compound under I in Example 1 (Scheme 1) using (3S*,4R*)-3-Benzyl-4-{[cyclopropyl-(2-oxo-1,2,3,4-tetrahydro-quinoline-4-carbonyl)-amino]-methyl}-pyrrolidine-1-carboxylic acid tert-butyl ester (100 mg, 0.19 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.35 min. MS (EI+): 422 [M+H]⁺

### Example 9

### A. (3S*,4R*)-3-Benzyl-4-[(3-tert-butoxycarbonyl-propylamino)-methyl]-pyrrolidine-1-carboxylic acid tert-butyl ester

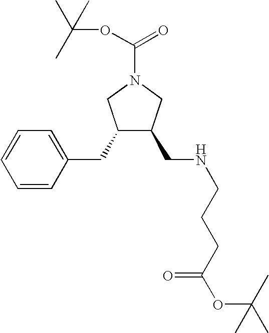

The title compound is prepared analogously as described for the title compound under G in Example 1 (Scheme 1) using (3S*,4S*)-3-Benzyl-4-formyl-pyrrolidine-1-carboxylic acid tert-butyl ester (200 mg, 0.69 mmol) and 3-Amino-butyric acid tert-butyl ester (121 mg, 0.76 mmol, Bachem).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.47 min. TLC, Rf (CH₂Cl₂/MeOH 19/1)=0.21, MS (EI+): 433 [M+H]⁺

### B. (3S*,4R*)-3-Benzyl-4-{[(3-tert-butoxycarbonyl-propyl)-(2-oxo-1,2,3,4-tetrahydro-quinoline-4-carbonyl)-amino]-methyl}-pyrrolidine-1-carboxylic acid tert-butyl ester

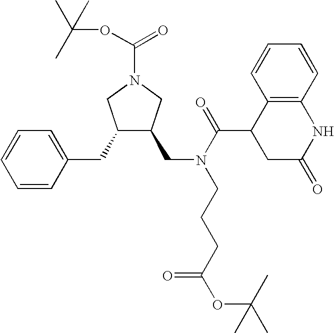

The title compound is prepared analogously as described for the title compound under H in Example 1 (Scheme 1) using (3S*,4R*)-3-Benzyl-4-[(3-tert-butoxycarbonyl-propylamino)-methyl]-pyrrolidine-1-carboxylic acid tert-butyl ester (313 mg, 0.72 mmol) and 2-Oxo-1,2,3,4-tetrahydro-quinoline-4-carboxylic acid (152 mg, 0.80 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.49 min. TLC, Rf (CH₂Cl₂/MeOH 95/5)=0.20, MS (EI+): 606 [M+H]⁺

### C. 4-[((3S*,4S*)-4-Benzyl-pyrrolidin-3-ylmethyl)-(2-oxo-1,2,3,4-tetrahydro-quinoline-4-carbonyl)-amino]-butyric acid

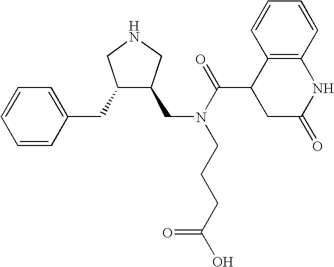

The title compound is prepared analogously as described for the title compound under I in Example 1 (Scheme 1) using (3S*,4R*)-3-Benzyl-4-{[(3-tert-butoxycarbonyl-propyl)-(2-oxo-1,2,3,4-tetrahydro-quinoline-4-carbonyl)-amino]-methyl}-pyrrolidine-1-carboxylic acid tert-butyl ester (361 mg, 0.60 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 2.56 and 2.72 min. MS (EI+): 450 [M+H]⁺

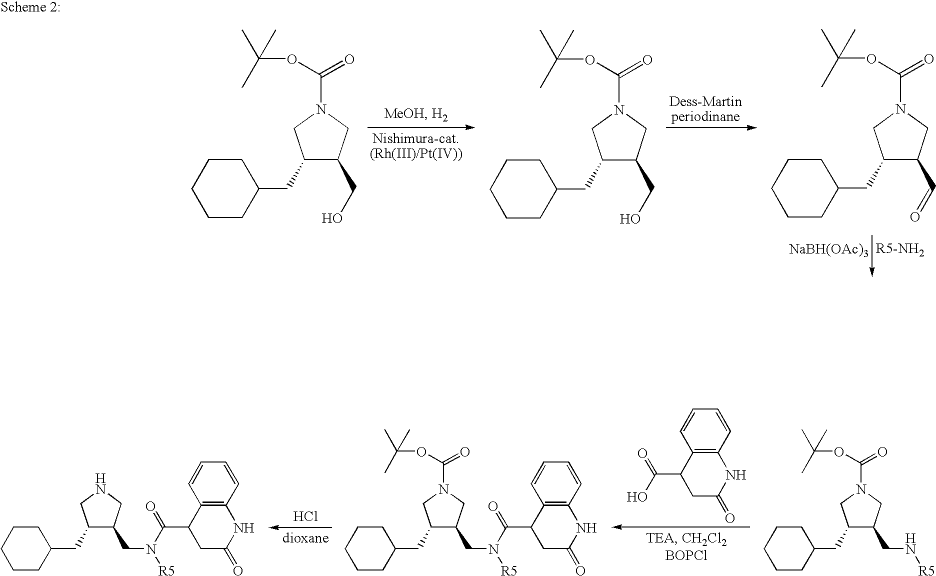

### Example 10

### 2-Oxo-1,2,3,4-tetrahydro-quinoline-4-carboxylic acid ((3S*,4S*)-4-cyclohexylmethyl-pyrrolidin-3-ylmethyl)-methyl-amide

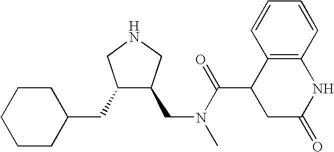

### A. (3S*,4S*)-3-Cyclohexylmethyl-4-hydroxymethyl-pyrrolidine-1-carboxylic acid tert-butyl ester

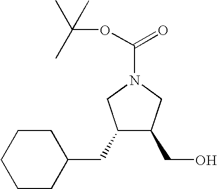

The title compound is prepared by hydrogenation of (3S*,4S*)-3-Benzyl-4-hydroxymethyl-pyrrolidine-1-carboxylic acid tert-butyl ester (3.0 g, 10.3 mmol) with Nishimura-catalyst.

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.40 min. TLC, Rf (CH₂Cl₂/MeOH 19/1)=0.26, MS (EI+): 298 [M+H]⁺

### B. (3S*,4S*)-3-Cyclohexylmethyl-4-formyl-pyrrolidine-1-carboxylic acid tert-butyl ester

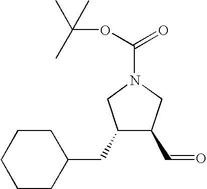

The title compound is prepared analogously as described for the title compound under F in Example 1 (Scheme 1) using (3S*,4S*)-3-Cyclohexylmethyl-4-hydroxymethyl-pyrrolidine-1-carboxylic acid tert-butyl ester (3.3 g, 11 mmol) and Dess-Martin Periodinane (9.4 g, 22 mmol, Rarechem).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.67 min. TLC, Rf (CH₂Cl₂/MeOH 19/1)=0.46

### C. (3S*,4R*)-3-Cyclohexylmethyl-4-methylaminomethyl-pyrrolidine-1-carboxylic acid tert-butyl ester

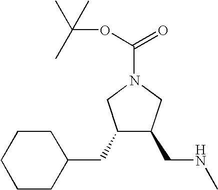

The title compound is prepared analogously as described for the title compound under G in Example 1 (Scheme 1) using (3S*,4S*)-3-Cyclohexylmethyl-4-formyl-pyrrolidine-1-carboxylic acid tert-butyl ester (200 mg, 0.68 mmol) and methylamine (0.84 mL, 6.8 mmol, 33% in EtOH)

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.52 min. TLC, Rf (CH₂Cl₂/MeOH 9/1)=0.17, MS (EI+): 311 [M+H]⁺

### D. (3S*,4R*)-3-Cyclohexylmethyl-4-{[methyl-(2-oxo-1,2,3,4-tetrahydro-quinoline-4-carbonyl)-amino]-methyl}-pyrrolidine-1-carboxylic acid tert-butyl ester

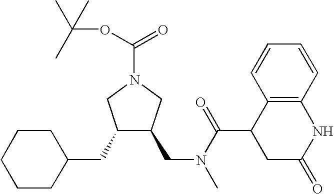

A mixture of (3S*,4R*)-3-Cyclohexylmethyl-4-methylaminomethyl-pyrrolidine-1-carboxylic acid tert-butyl ester (0.39 g, 0.89 mmol), BOP-Cl (0.15 g, 0.57 mmol), NEt₃ (0.27 mL, 1.9 mmol) and 2-Oxo-1,2,3,4-tetrahydro-quinoline-4-carboxylic acid (80 mg, 0.42 mmol) in CH₂Cl₂ (10 mL) is refluxed for 2.5 h. The reaction mixture is quenched with water extracted with CH₂Cl₂, dried with Na₂SO₄, filtered and the solvent is removed in vacuo. The crude product is purified by flash chromatography on silica gel (eluent; CH₂Cl₂/MeOH 19/1) to give the title compound.

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.48 min. TLC, Rf (CH₂Cl₂/MeOH 19/1)=0.25, MS (EI+): 484 [M+H]⁺

### E. 2-Oxo-1,2,3,4-tetrahydro-quinoline-4-carboxylic acid ((3S*,4S*)-4-cyclohexyl-methyl-pyrrolidin-3-ylmethyl)-methyl-amide

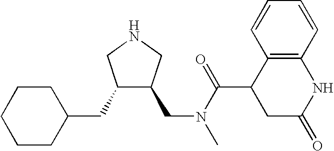

The title compound is prepared analogously as described for the title compound under I in Example 1 (Scheme 1) using (3S*,4R*)-3-Cyclohexylmethyl-4-{[methyl-(2-oxo-1,2,3,4-tetrahydro-quinoline-4-carbonyl)-amino]-methyl}-pyrrolidine-1-carboxylic acid tert-butyl ester (143 mg, 0.30 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.40 min. TLC, Rf (CH₂Cl₂/MeOH/H₂O/AcOH 90/10/1/0.5)=0.14. MS (EI+): 384 [M+H]⁺

### Example 11

### 4-[((3S*,4S*)-4-Cyclohexylmethyl-pyrrolidin-3-ylmethyl)-(2-oxo-1,2,3,4-tetrahydro-quinoline-4-carbonyl)-amino]-butyric acid

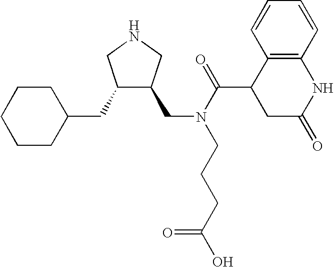

### A. (3R*,4S*)-3-[(3-tert-Butoxylcarbonyl-propylamino)-methyl]-4-cyclohexylmethyl-pyrrolidine-1-carboxylic acid tert-butyl ester

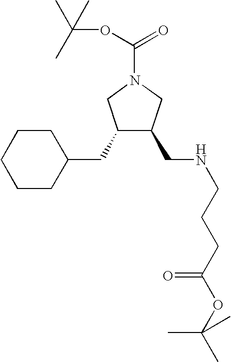

The title compound is prepared analogously as described for the title compound under C in Example 10 (Scheme 2) using (3S*,4S*)-3-Cyclohexylmethyl-4-formyl-pyrrolidine-1-carboxylic acid tert-butyl ester (265 mg, 0.90 mmol) and 3-Amino-butyric acid tert-butyl ester (157 mg, 0.99 mmol, Bachem).

TLC, Rf (CH₂Cl₂/MeOH 19/1)=0.14, MS (EI+): 439 [M]⁺

### B. (3R*,4S*)-3-{[(3-tert-Butoxycarbonyl-propyl)-(2-oxo-1,2,3,4-tetrahydro-quinoline-4-carbonyl)-amino]-methyl}-4-cyclohexylmethyl-pyrrolidine-1-carboxylic acid tert-butyl ester

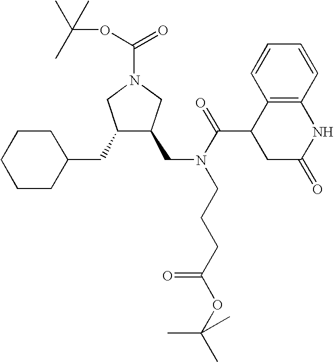

The title compound is prepared analogously as described for the title compound under D in Example 10 (Scheme 2) using (3R*,4S*)-3-[(3-tert-Butoxycarbonyl-propylamino)-methyl]-4-cyclohexylmethyl-pyrrolidine-1-carboxylic acid tert-butyl ester (166 mg, 0.38 mmol) and 2-Oxo-1,2,3,4-tetrahydro-quinoline-4-carboxylic acid (80 mg, 0.42 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 6.28 min. TLC, Rf (CH₂Cl₂/MeOH 19/1)=0.24, MS (EI+): 612 [M]⁺

### C. 4-[((3S*,4S*)-4-Cyclohexylmethyl-pyrrolidin-3-ylmethyl)-(2-oxo-1,2,3,4-tetrahydro-quinoline-4-carbonyl)-amino]-butyric acid

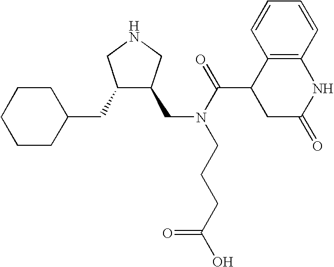

The title compound is prepared analogously as described for the title compound under I in Example 1 (Scheme 1) using (3R*,4S*)-3-{[(3-tert-Butoxycarbonyl-propyl)-(2-oxo-1,2,3,4-tetrahydro-quinoline-4-carbonyl)-amino]-methyl}-4-cyclohexylmethyl-pyrrolidine-1-carboxylic acid tert-butyl ester (203 mg, 0.33 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.25 min. TLC, Rf (CH₂Cl₂/MeOH/H₂O/AcOH 75/27/5/0.5)=0.45. MS (EI+): 456 [M+H]⁺

### Example 12

### 2-Oxo-1,2,3,4-tetrahydro-quinoline-4-carboxylic acid ((3S*,4S*)-4-cyclohexylmethyl-pyrrolidin-3-ylmethyl)-cyclopropyl-amide

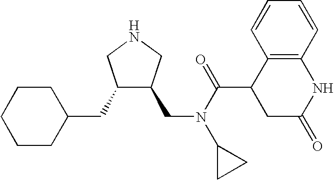

### A. (3S*,4R*)-3-Cyclohexylmethyl-4-cyclopropylaminomethyl-pyrrolidine-1-carboxylic acid tert-butyl ester

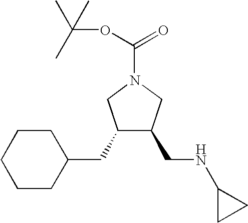

The title compound is prepared analogously as described for the title compound under G in Example 1 (Scheme 1) using (3S*,4S*)-3-Cyclohexylmethyl-4-formyl-pyrrolidine-1-carboxylic acid tert-butyl ester (500 mg, 1.7 mmol) and cyclopropylamine (148 mg, 2.5 mmol). TLC, Rf (CH₂Cl₂/MeOH 19/1)=0.22, MS (EI+): 337 [M+H]⁺

### B. (3S*,4R*)-3-Cyclohexylmethyl-[cyclopropyl-(2-oxo-1,2,3,4-tetrahydro-quinoline-4-carbonyl)-amino]-methyl)-pyrrolidine-1-carboxylic acid tert-butyl ester

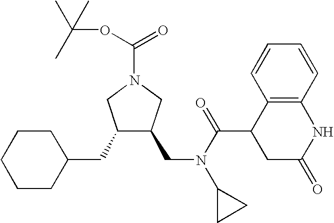

The title compound is prepared analogously as described for the title compound under D in Example 10 (Scheme 2) using (3S*,4R*)-3-Cyclohexylmethyl-4-cyclopropylaminomethyl-pyrrolidine-1-carboxylic acid tert-butyl ester (80 mg, 0.24 mmol) and 2-Oxo-1,2,3,4-tetrahydro-quinoline-4-carboxylic acid (60 mg, 0.31 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.81 min. TLC, Rf (CH₂Cl₂/MeOH 19/1)=0.28, MS (EI+): 510 [M+H]⁺

### C. 2-Oxo-1,2,3,4-tetrahydro-quinoline-4-carboxylic acid ((3S*,4S*)-4-cyclohexylmethyl-pyrrolidin-3-ylmethyl)-cyclopropyl-amide

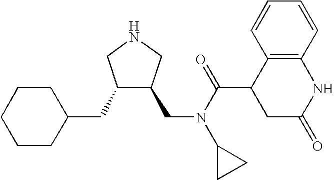

The title compound is prepared analogously as described for the title compound under D in Example 1 (Scheme 1) using (3S*,4R*)-3-Cyclohexylmethyl-4-{[cyclopropyl-(2-oxo-1,2,3,4-tetrahydro-quinoline-4-carbonyl)-amino]-methyl}-pyrrolidine-1-carboxylic acid tert-butyl ester (54 mg, 0.11 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.76 min. TLC, Rf (CH₂Cl₂/MeOH/H₂O/AcOH 90/10/1/0.5)=0.18. MS (EI+): 410 [M+H]⁺

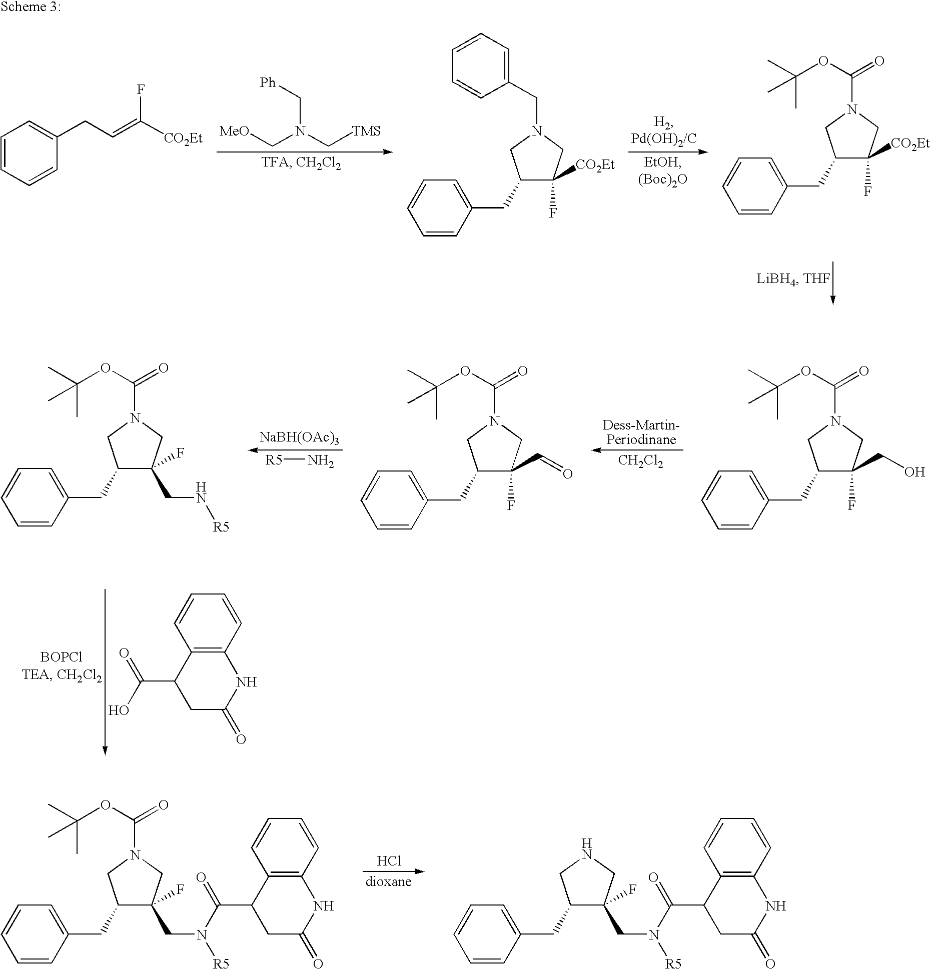

### Example 13

### rac-2-Oxo-1,2,3,4-tetrahydro-quinoline-4-carboxylic acid ((3R,4R)-4-benzyl-3-fluoro-pyrrolidin-3-ylmethyl)-cyclopropyl-amide

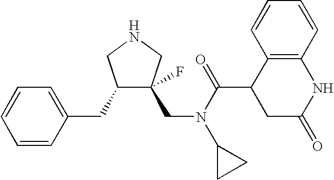

### A. rac-(3R,4R)-1,4-Dibenzyl-3-fluoro-pyrrolidine-3-carboxylic acid ethyl ester

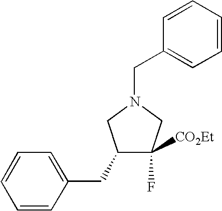

To a solution of (Z)-2-Fluoro-4-phenyl-but-2-enoic acid ethyl ester (prepared according to Tetrahedron 1991, 27, 4905) (2.5 g, 12 mmol) and N-Benzyl-N-(methoxymethyl) trimethylsilylamine (3.9 g, 16 mmol) in 50 mL CH₂Cl₂ is added TFA (0.020 mL). After 2 h at ambient temperature, the solvent is removed in vacuo and the product is purified by flash chromatography on silica gel (eluent; PE/EtOAc 10/1) to give the title compound.

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.79 min. TLC, Rf (PE/EtOAc 10/1)=0.15, MS (EI+): 342 [M+H]⁺

### B. rac-(3R,4R)-4-Benzyl-3-fluoro-pyrrolidine-1,3-dicarboxylic acid 1-tert-butyl ester 3-ethyl ester

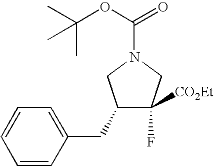

The title compound is prepared analogously as described for the title compound under D in Example 1 (Scheme 1) using rac-(3R,4R)-1,4-Dibenzyl-3-fluoro-pyrrolidine-3-carboxylic acid ethyl ester (3.8 g, 11 mmol) and (BOC)₂O (2.9 g, 13 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 5.39 min. TLC, Rf (PE/EtOAc 10/1)=0.27, MS (EI+): 296 [M-55]⁺

### C. rac-3R,4R)-4-Benzyl-3-fluoro-3-hydroxymethyl-pyrrolidine-1-carboxylic acid tert-butyl ester

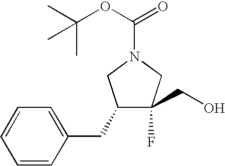

To a solution of rac-(3R,4R)-4-Benzyl-3-fluoro-pyrrolidine-1,3-dicarboxylic acid 1-tert-butyl ester 3-ethyl ester (2.7 g, 7.8 mmol) in THF (20 mL) is added at 0° C. LiBH₄ (8 mmol, 4 mL of 2M solution in THF). After stirring for 2 h at ambient temperature the reaction mixture is quenched by careful addition of 0.5 M NaOH (40 mL). extracted with TBME, dried over MgSO₄, filtered and the solvent is removed in vacuo to give the title compound, which is used without further purification.

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.43 min. TLC, Rf (PE/EtOAc 2/1)=0.6, MS (EI+): 254 [M-55]⁺

### D. rac-(3R,4R)-4-Benzyl-3-fluoro-3-formyl-pyrrolidine-1-carboxylic acid tert-butyl ester

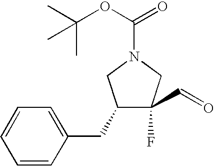

The title compound is prepared analogously as described for the title compound under F in Example 1 (Scheme 1) using rac-(3R,4R)-4-Benzyl-3-fluoro-3-hydroxymethyl-pyrrolidine-1-carboxylic acid tert-butyl ester (200 mg, 0.65 mmol) and Dess-Martin periodinane (331 mg, 0.78 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 4.13 min. TLC, Rf (CH₂Cl₂/MeOH 95/5)=0.24.

### E. rac-(3S,4R)-4-Benzyl-3-cyclopropylaminomethyl-3-fluoro-pyrrolidine-1-carboxylic acid tert-butyl ester

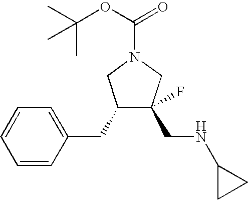

The title compound is prepared analogously as described for the title compound under G in Example 1 (Scheme 1) using rac-(3R,4R)-4-Benzyl-3-fluoro-3-formyl-pyrrolidine-1-carboxylic acid tert-butyl ester (200 mg, 0.65 mmol), cyclopropylamine (74 mg, 1.3 mmol) and NaBH(OAc)₃ (290 mg, 1.3 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.91 min. TLC, Rf (CH₂Cl₂/MeOH 95/5)=0.50. MS (EI+): 349 [M+H]⁺

### E. rac-(3S,4R)-4-Benzyl-3-{[cyclopropyl-(2-oxo-1,2,3,4-tetrahydro-quinoline-4-carbonyl)-amino]-methyl}-3-fluoro-pyrrolidine-1-carboxylic acid tert-butyl ester

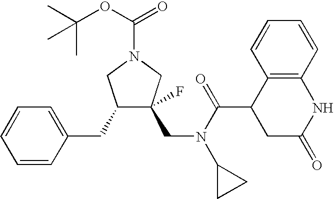

The title compound is prepared analogously as described for the title compound under D in Example 10 (Scheme 2) using rac-(3S,4R)-4-Benzyl-3-cyclopropylaminomethyl-3-fluoro-pyrrolidine-1-carboxylic acid tert-butyl ester (160 mg, 0.46 mmol) and 2-Oxo-1,2,3,4-tetrahydro-quinoline-4-carboxylic acid (105 mg, 0.6 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mt/min): 5.07 min. MS (EI+): 522 [M+H]⁺

### F. rac-2-Oxo-1,2,3,4-tetrahydro-quinoline-4-carboxylic acid ((3R,4R)-4-benzyl-3-fluoro-pyrrolidin-3-ylmethyl)-cyclopropyl-amide

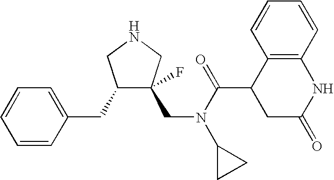

The title compound is prepared analogously as described for the title compound under I in Example 1 (Scheme 1) using rac-(3S,4R)-4-Benzyl-3-{[cyclopropyl-(2-oxo-1,2,3,4-tetrahydro-quinoline-4-carbonyl)-amino]-methyl}-3-fluoro-pyrrolidine-1-carboxylic acid tert-butyl ester (160 mg, 0.31 mmol).

t_{R} (HPLC, Nucleosil C18 column, 20-100% CH₃CN/H₂O/6 min, 100% CH₃CN/1.5 min, 100-20% CH₃CN/H₂O/0.5 min, CH₃CN and H₂O containing 0.1% TFA, flow: 1.0 mL/min): 3.26 & 3.44 min. MS (EI+): 422 [M+H]⁺

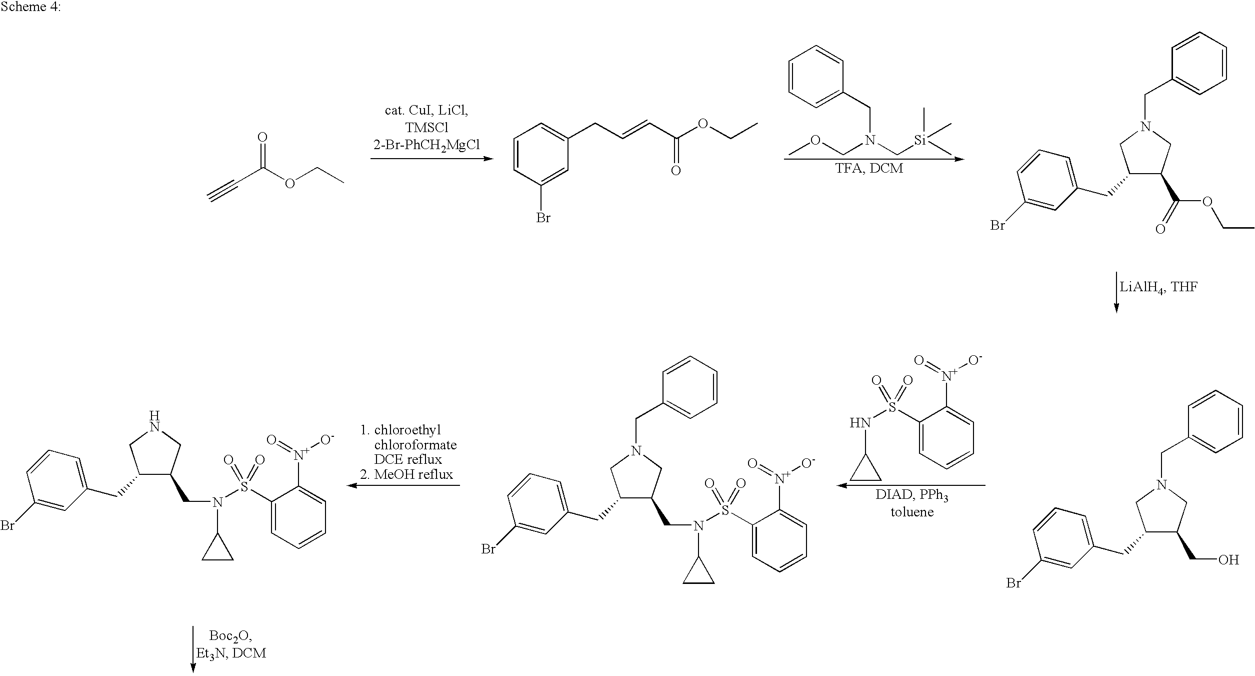

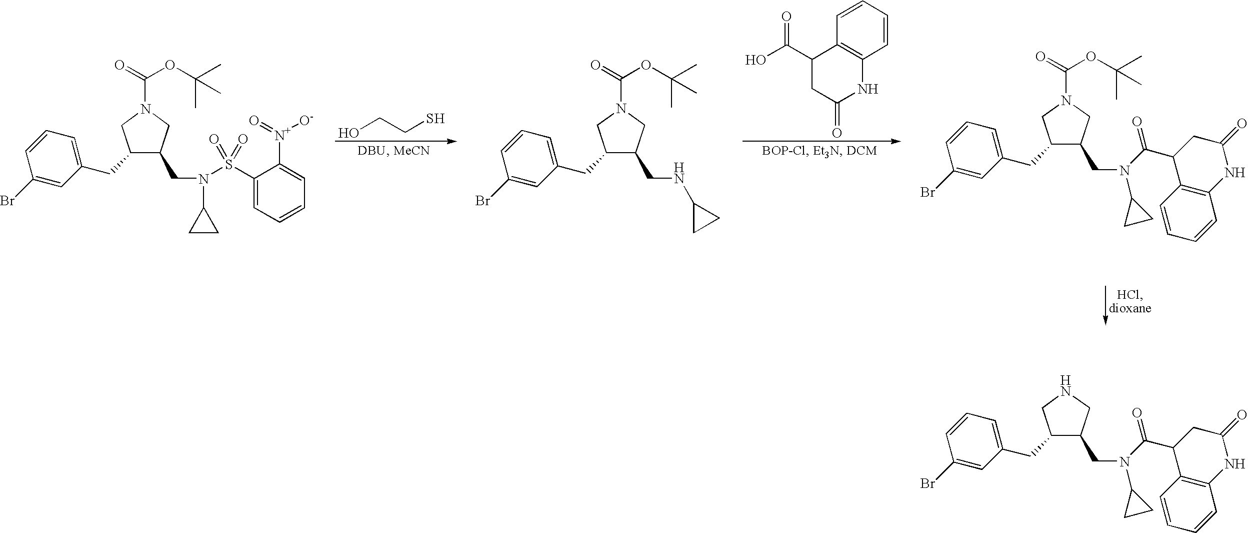

### Example 14

### (R)-2-Oxo-1,2,3,4-tetrahydro-quinoline-4-carboxylic acid [(3S*,4S*)-4-(3-bromo-benzyl)-pyrrolidin-3-ylmethyl]-cyclopropyl-amide

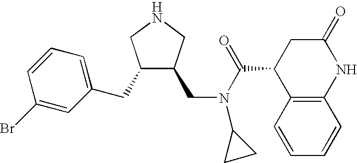

### A. (E)-4-(3-Bromo-phenyl)-but-2-enoic acid ethyl ester

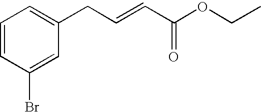

CuI (1 g, 5.15 mmol) and LiCl (437 mg, 10.3 mmol) is placed in a 250 mL round bottom flask under Ar. Dry THF (100 mL) is added to these salts, and the mixture is stirred at rt for a period of 0.5 h until complete dissolution has occurred. The clear, light yellow homogeneous solution is cooled to −78° C. and ethyl propriolate (1.2 mL, 10.3 mmol) is added, followed by TMSCl (1.73 mL, 13.4 mmol). After 5 min at −78° C., 50 mL (12.4 mmol) of a 2M solution of 3-bromo benzylmagnesium bromide in Et₂O is added dropwise with a canula, and the solution is stirred at −78° C. for 1 hour. Saturated ammonium chloride solution is added to quench the reaction at −78° C., and the mixture is allowed to warm to rt and to stir for 30 min. The product is extracted with Et₂O (3×100 mL) and washed with water followed by brine. The organic layers are combined and dried with Na₂SO₄, filtered and concentrated in vacuo to give the crude product. Column chromatography on silica of the crude product (20% ethyl acetate in cyclohexane) affords the diastereoselective pure (E)-4-(3-Bromo-phenyl)-but-2-enoic acid ethyl ester. MS (ESI): 286 and 287 [M+H₂O]⁺.

### B. rac-(3S,4S)-1-Benzyl-4-(3-bromo-benzyl)-pyrrolidine-3-carboxylic acid ethyl ester

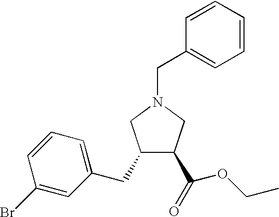

To a solution of (E)-4-(3-Bromo-phenyl)-but-2-enoic acid ethyl ester (4.5 g, 16.7 mmol) in DCM (50 ml) is added N-Benzyl-N-(methoxymethyl) trimethylsilylamine (5.56 mL, 21.7 mmol) and TFA (90 L, 1.17 mmol). The mixture is stirred at rt for 15 min and concentrated in vacuo. Column chromatography on silica of the crude product (20% ethyl acetate in cyclohexane) affords a racemic mixture of the title compound. MS (ESI): 403 and 404 [M+H]⁺.

### C. rac-[(3S,4S)-1-Benzyl-4-(3-bromo-benzyl)-pyrrolidin-3-yl]-methanol

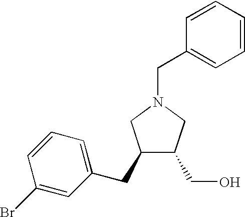

A solution of rac-(3S,4S)-1-Benzyl-4-(3-bromo-benzyl)-pyrrolidine-3-carboxylic acid ethyl ester (5 g, 12.4 mmol) in THF (100 mL) is cooled at 0° C. and LiAlH₄ (486 mg, 12.4 mmol) is added portionwise. The mixture is stirred at 0° C. for 1 hour and poured into 5% aq NaOH (250 mL) then filtered over Celite. The filtrate is diluted with water (200 mL) and extracted with EtOAc (3×150 mL). The organic phases are combined, washed with water and brine, dried over Na₂SO₄, filtered and concentrated in vacuo to give the title compound as a racemic mixture This crude mixture was used for the next step without further purification. MS (ESI): 360 and 361 [M+H]⁺.

### D. rac-N-[(3S,4S)-1-Benzyl-4-(3-bromo-benzyl)-pyrrolidin-3-ylmethyl]-N-cyclopropyl-2-nitro-benzenesulfonamide

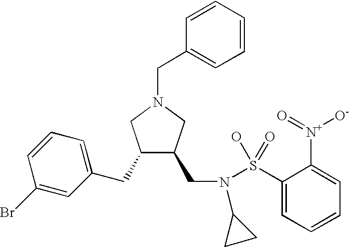

To a solution of rac-N-[(3S,4S)-1-Benzyl-4-(3-bromo-benzyl)-pyrrolidin-3-yl]-methanol (1 g, 2.78 mmol) in toluene is added PPh₃ (1.09 g, 4.16 mmol). The reaction mixture is cooled under argon at 0° C. then DIAD (820 L, 4.16 mmol) is added. The reaction mixture is allowed to warm to rt stirred overnight and concentrated in vacuo. Column chromatography on silica of the crude product (30% ethyl acetate in cyclohexane) affords the title compound as a racemic mixture. MS (ESI): 585 and 586 [M+H]⁺.

### E. rac-N-[(3S,4S)-4-(3-bromo-benzyl)-pyrrolidin-3-ylmethyl]-N-cyclopropyl-2-nitro-benzenesulfonamide

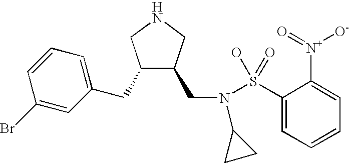

To a solution of rac-N-[(3S,4S)-1-Benzyl-4-(3-bromo-benzyl)-pyrrolidin-3-ylmethyl]-N-cyclopropyl-2-nitro-benzenesulfonamide (800 mg, 1.37 mmol) in DCE (20 mL) is added 1-chloroethyl chloroformate (298 L, 2.74 mmol). The mixture is refluxed overnight and concentrated in vacuo. The residue is dissolved in MeOH (20 mL), refluxed for 1 hour and concentrated in vacuo. Column chromatography on silica of the crude product (5% MeOH in DCM) affords the title compound as a racemic mixture. MS (ESI): 496 and 497 [M+H]⁺.

### F. rac-N-[(3S,4S)-1-carboxylic acid tert-butyl ester-4-(3-bromo-benzyl)-pyrrolidin-3-ylmethyl]-N-cyclopropyl-2-nitro-benzenesulfonamide

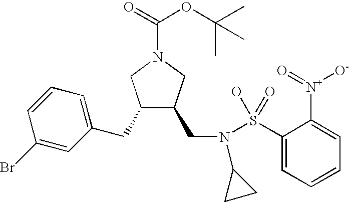

To a solution of rac-N-[(3S,4S)-4-(3-bromo-benzyl)-pyrrolidin-3-ylmethyl]-N-cyclopropyl-2-nitro-benzenesulfonamide (500 mg, 1.01 mmol) in DCM (20 mL) is added Et₃N (397 μL, 2.02 mmol) and (BOC)₂O (441 mg, 2.02 mmol). The reaction mixture is concentrated in vacuo. Column chromatography on silica of the crude product (30% EtOAc in Cyclohexane) affords the title compound as a racemic mixture. MS (ESI): 538 and 539 [M-tBu]⁺.

### G. rac-N-[(3S,4S)-1-carboxylic acid tert-butyl ester-4-(3-bromo-benzyl)-pyrrolidin-3-ylmethyl]-N-cyclopropyl

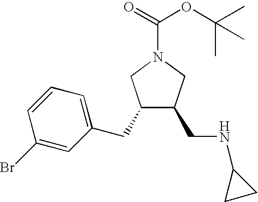

To a solution of rac-N-[(3S,4S)-1-carboxylic acid tert-butyl ester-4-(3-bromo-benzyl)-pyrrolidin-3-ylmethyl]-N-cyclopropyl-2-nitro-benzenesulfonamide (530 mg, 0.891 mmol) in MeCN (20 mL) is added DBU (667 μL, 4.46 mmol) and 2-mercapto ethanol (687 μL, 9.81 mmol). The mixture is stirred at rt for 1 hour and the solvent is removed in vacuo. The residue is quenched with water (100 mL), extracted with DCM (100 mL), washed with brine, dried over Na₂SO₄, filtered and concentrated. The residue is purified on Reverse Phase HPLC affording the title compound as a racemic mixture. MS (ESI): 409 and 411 [M+H]⁺.

### H. rac-(3S,4R)-3-(3-Bromo-benzyl)-4-{[cyclopropyl-(2-oxo-1,2,3,4-tetrahydro-quinoline-4-carbonyl)-amino]-methyl}-pyrrolidine-1-carboxylic acid tert-butyl ester

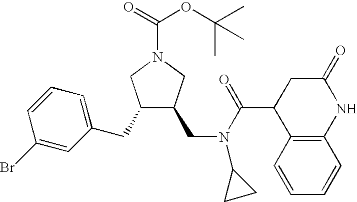

To a solution of rac-N-[(3S,4S)-1-carboxylic acid tert-butyl ester-4-(3-bromo-benzyl)-pyrrolidin-3-ylmethyl]-N-cyclopropyl (350 mg, 0.855 mmol) in DCM (20 mL) is added 2-oxo-tetrahydroquinoline-4-carboxylic acid (196 mg, 1.03 mmol), BOP-Cl (337 mg, 1.28 mmol) and Et₃N (594 L, 4.27 mmol). The mixture is refluxed under argon for 2 hours and concentrated in vacuo. The residue is extracted with EtOAc (50 mL), washed with water and brine, dried over Na₂SO₄, filtered and concentrated. The residue is purified on Reverse Phase HPLC affording the title compound as a diasteromeric mixture. MS (ESI): 409 and 411 [M+H]⁺.

### I. (S)-2-Oxo-1,2,3,4-tetrahydro-quinoline-4-carboxylic acid [(3S*,4S*)-4-(3-bromo-benzyl)-pyrrolidin-3-ylmethyl]-cyclopropyl-amide

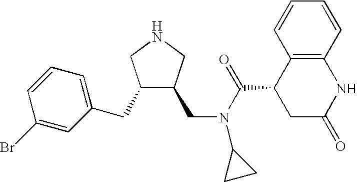

To a solution of (3S,4R)-3-(3-Bromo-benzyl)-4-{[cyclopropyl-(2-oxo-1,2,3,4-tetrahydro-quinoline-4-carbonyl)-amino]-methyl}-pyrrolidine-1-carboxylic acid tert-butyl ester (220 mg, 0.38 mmol) in MeOH (10 ml) is added a solution of 4N HCl in dioxane (1.89 ml, 7.55 mmol).

The mixture is stirred at rt for 15 min followed by concentration in vacuo. The residue is purified on Reverse Phase HPLC. MS (EI+): 406 [M+H]⁺.

The diastereomeric mixture is separated by Chiral HPLC (Chiralpak AD-H 0.46×25 cm, EtOH 100%+0.05% DEA, flow 0.8 mL/min, detector UV 210 nM) to give to give the title compound as a single diastereoisomer. t_{R}: 10.63 min. The three others isomers, t_{R}: 8.76, 18.85 and 21.69 min

### Example 15

### (R)-2-Oxo-1,2,3,4-tetrahydro-quinoline-4-carboxylic acid [(3S*,4S*)-4-(2-bromo-benzyl)-pyrrolidin-3-ylmethyl]-cyclopropyl-amide

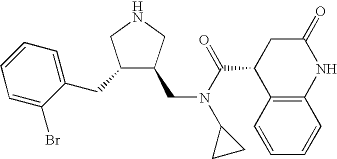

### A. (E)-4-(2-Bromo-phenyl)-but-2-enoic acid ethyl ester

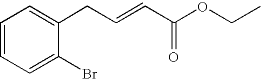

The title compound is prepared analogously as described for the title compound under A in Example 14 (Scheme 4) using 50 mL (12.4 mmol) of a 2M solution of 2-bromo benzylmagnesium bromide in Et₂O. MS (ESI): 286 and 287 [M+H₂O]⁺.

### B. rac-(3S,4S)-1-Benzyl-4-(2-bromo-benzyl)-pyrrolidine-3-carboxylic acid ethyl ester

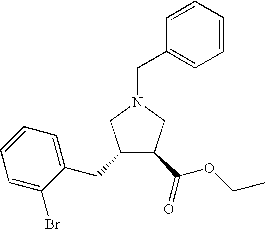

The title compound is prepared analogously as described for the title compound under B in Example 14 (Scheme 4) using (E)-4-(2-bromo-phenyl)-but-2-enoic acid ethyl ester (2.4 g, 8.92 mmol). MS (ESI): 403 and 404 [M+H]⁺.

### C. rac-[(3S,4S)-1-Benzyl-4-(2-bromo-benzyl)-pyrrolidin-3-yl]-methanol

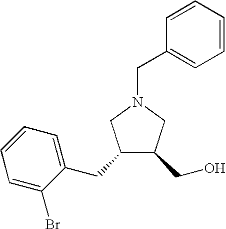

The title compound is prepared analogously as described for the title compound under C in Example 14 (Scheme 4) using a solution of rac-(3S,4S)-1-Benzyl-4-(2-bromo-benzyl)-pyrrolidine-3-carboxylic acid ethyl ester (3.5 g, 8.7 mmol) in THF (100 mL). MS (ESI): 360 and 361 [M+H]⁺.

### D. rac-N-[(3S,4S)-1-Benzyl-4-(2-bromo-benzyl)-pyrrolidin-3-ylmethyl]-N-cyclopropyl-2-nitro-benzenesulfonamide

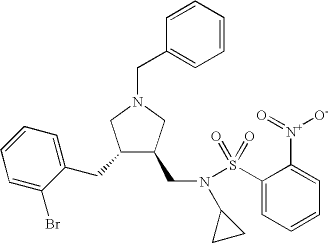

The title compound is prepared analogously as described for the title compound under D in Example 14 (Scheme 4) using a solution of raq-[(3S,4S)-1-Benzyl-4-(2-bromo-benzyl)-pyrrolidin-3-yl]-methanol (3.1 g, 8.6 mmol) in toluene. MS (ESI): 585 and 586 [M+H]⁺.

### E. rac-N-[(3S,4S)-4-(2-bromo-benzyl)-pyrrolidin-3-ylmethyl]-N-cyclopropyl-2-nitro-benzenesulfonamide

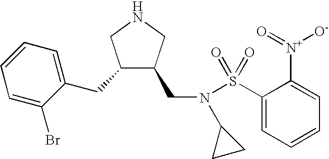

The title compound is prepared analogously as described for the title compound under E in Example 14 (Scheme 4) using a solution of rac-N-[(3S,4S)-1-benzyl-4-(2-bromo-benzyl)-pyrrolidin-3-ylmethyl]-N-cyclopropyl-2-nitro-benzenesulfonamide (1 g, 1.71 mmol) in DCE (20 mL). MS (ESI): 496 and 497 [M+H]⁺.

### F. rac-N-[(3S,4S)-1-carboxylic acid tert-butyl ester-4-(2-bromo-benzyl)-pyrrolidin-3-ylmethyl]-N-cyclopropyl-2-nitro-benzenesulfonamide

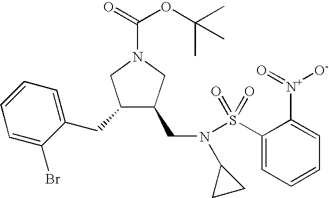

The title compound is prepared analogously as described for the title compound under F in Example 14 (Scheme 4) using a solution of rac-N-[(3S,4S)-4-(2-bromo-benzyl)-pyrrolidin-3-ylmethyl]-N-cyclopropyl-2-nitro-benzenesulfonamide (500 mg, 1.01 mmol) in DCM (20 mL). MS (ESI): 538 and 539 [M-tBu]⁺.

### G. rac-N-[(3S,4S)-1-carboxylic acid tert-butyl ester-4-(2-bromo-benzyl)-pyrrolidin-3-ylmethyl]-N-cyclopropyl

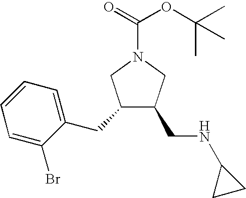

The title compound is prepared analogously as described for the title compound under G in Example 14 (Scheme 4) using a solution of rac-N-[(3S,4S)-1-carboxylic acid tert-butyl ester-4-(2-bromo-benzyl)-pyrrolidin-3-ylmethyl]-N-cyclopropyl-2-nitro-benzenesulfonamide (560 mg, 0.94 mmol) in MeCN (20 mL). MS (ESI): 409 and 411 [M+H]⁺.

### H. (3S,4R)-3-(2-Bromo-benzyl)-4-{[cyclopropyl-(2-oxo-1,2,3,4-tetrahydro-quinoline-4-carbonyl)-amino]-methyl}-pyrrolidine-1-carboxylic acid tert-butyl ester

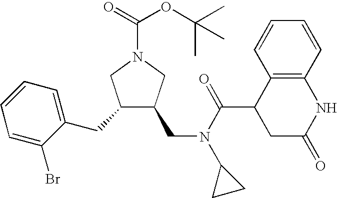

The title compound is prepared analogously as described for the title compound under H in Example 14 (Scheme 4) using a solution of rac-N-[(3S,4S)-1-carboxylic acid tert-butyl ester-4-(2-bromo-benzyl)-pyrrolidin-3-ylmethyl]-N-cyclopropyl (340 mg, 0.83 mmol) and 2-oxo-tetrahydroquinoline-4-carboxylic acid (191 mg, 1 mmol) in DCM (20 mL). MS (ESI): 409 and 411 [M+H]⁺.

### I. (S)-2-Oxo-1,2,3,4-tetrahydro-quinoline-4-carboxylic acid [(3S*,4S*)-4-(2-bromo-benzyl)-pyrrolidin-3-ylmethyl]-cyclopropyl-amide

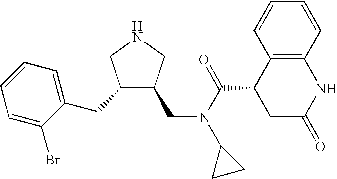

The title compound is prepared analogously as described for the title compound under I in Example 1 (Scheme 1) using a solution of rac-N-[(3S,4S)-1-carboxylic acid tert-butyl ester-4-(2-bromo-benzyl)-pyrrolidin-3-ylmethyl]-N-cyclopropyl (220 mg, 0.38 mmol) in MeOH (10 ml). MS (EI+): 406 [M+H]⁺.

The diastereomeric mixture is separated by Chiral HPLC (Chiralcel AD-H 0.30×25 cm, MeOH 40%+0.2% IPAm, flow 100 g/min, detector UV 220 nM) to give to give the title compound as a single diastereoisomer. t_{R}: 3.6 min. The three others isomers, t_{R}: 5.15, 6.22 and 14.48 min

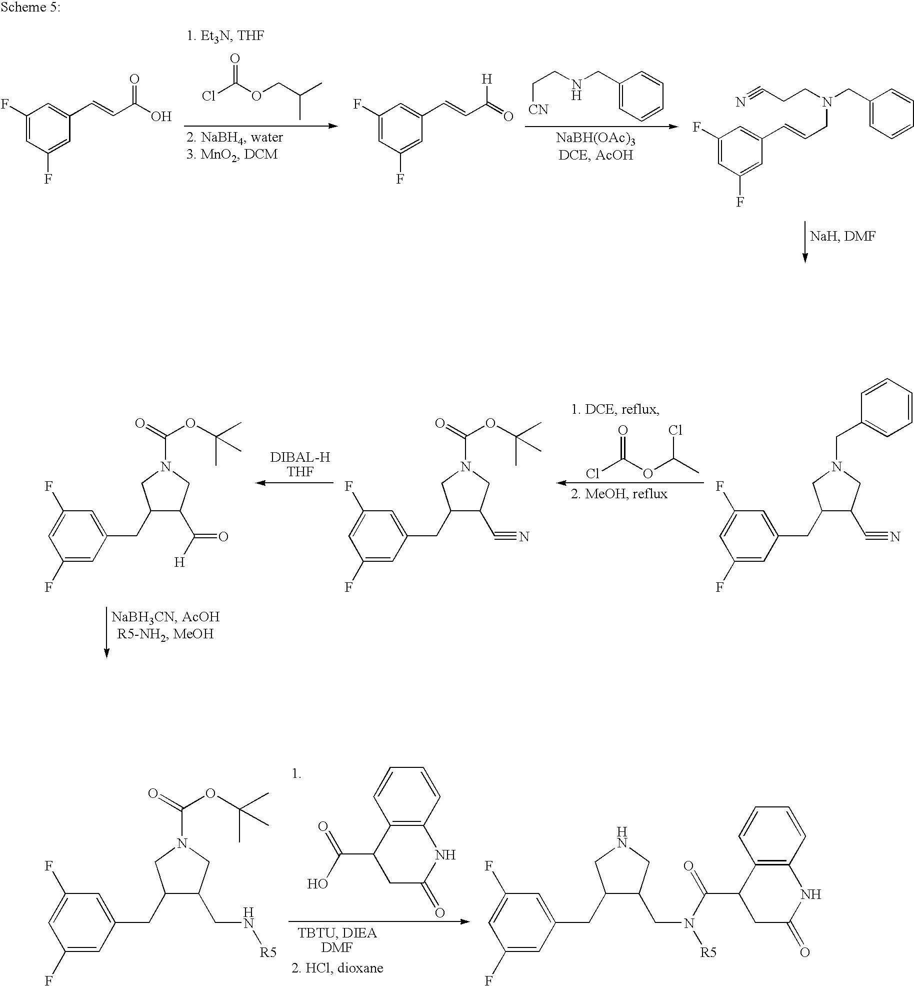

### Example 16

### 4-[[4-(3,5-Difluoro-benzyl)-pyrrolidin-3-ylmethyl]-(2-oxo-1,2,3,4-tetrahydro-quinoline-4-carbonyl)-amino]-butyric acid

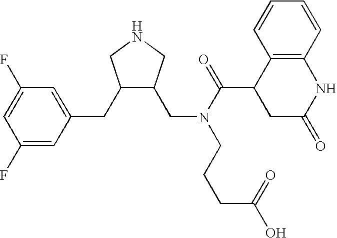

### A. (E)-3-(3,5-Difluoro-phenyl)-prop-2-en-1-ol

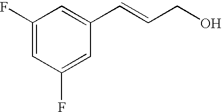

To a cold (0° C.) solution of 3,5-difluorophenyl (4 g, 21.7 mmoles) and Et₃N (3.62 mL, 26.1 mmol) in THF (50 mL) is added dropwise isobutyl chloroformate (3.24 mL, 25 mmol). A white precipitate appeared. The mixture is stirred for 1 hour, filtered and the filtrate is diluted with ice/water (10 ml). NaBH₄ (2.05 g, 54.3 mmol) is added and the mixture is stirred for 3 hours at rt. The reaction mixture is acidified with HCl 2N and extracted with DCM, washed with brine, dried over Na₂SO₄, filtered and concentrated. Column chromatography on silica of the crude product (30% ethyl acetate in cyclohexane) affords (E)-3-(3,5-Difluoro-phenyl)-prop-2-en-1-ol as a colourless syrup. MS (EI+): 169 [M−H]⁺

### B. (E)-3-(3,5-Difluoro-phenyl)-propenal

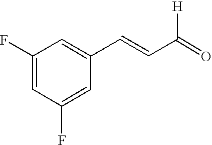

To a solution of (E)-3-(3,5-Difluoro-phenyl)-prop-2-en-1-ol (1.8 g, 10.6 mmol) in DCM (25 mL) is added MnO₂ (5.78 g, 42.3 mmol). The mixture is stirred at rt overnight, filtered over glass-fiber and concentrated to give (E)-3-(3,5-Difluoro-phenyl)-propenal as a white powder which is used without further purification in the next step. MS (EI+): 168 [M−H]⁺

### C. 3-{Benzyl-[(E)-3-(3,5-difluoro-phenyl)-allyl]-amino}-propionitrile

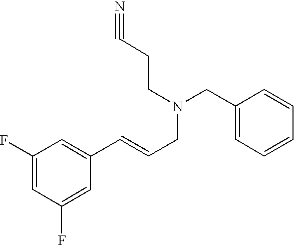

(E)-3-(3,5-Difluoro-phenyl)-propenal (2.5 g, 14.9 mmol) and 3-(benzylamino) propionitrile (3.49 mL, 22.3 mmol) are mixed at rt in DCE (100 mL) for 15 min then NaBH(OAc)₃ (7.88 g, 37.2 mmol) and AcOH (1.17 mL, 14.9 mmol) is added. After stirring overnight at rt, the reaction mixture is quenched carefully with sat aq NaHCO₃ and extracted with DCM (2×150 mL). The combined organic phases are washed with water, brine, dried over Na₂SO₄, filtered and concentrated. Column chromatography on silica of the crude product (20% ethyl acetate in cyclohexane) affords 3-{Benzyl-[(E)-3-(3,5-difluoro-phenyl)-allyl]-amino}-propionitrile as a colourless syrup. MS (EI+): 313 [M+H]⁺

### D. rac-1-Benzyl-4-(3,5-difluoro-benzyl)-pyrrolidine-3-carbonitrile

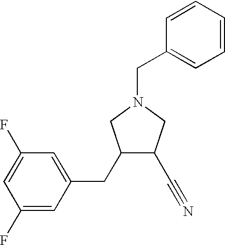

To a solution of 3-{Benzyl-[(E)-3-(3,5-difluoro-phenyl)-allyl]-amino}-propionitrile (4 g, 12.8 mmol) in DMF (128 mL) at rt under argon is added NaH (1.02 gL of 60% in mineral oil, 25.6 mmol). The mixture is stirred at rt for 4 hours, poured into ice and sat aq NaHCO₃ and extracted with EtOAc (200 mL). The organic phase is washed with water and brine, dried over Na₂SO₄, filtered and concentrated. Column chromatography on silica of the crude product (20% ethyl acetate in cyclohexane) affords the title compound as a light yellow syrup. MS (EI+): 313 [M+H]⁺

### E. rac-4-(3,5-Difluoro-benzyl)-pyrrolidine-3-carbonitrile

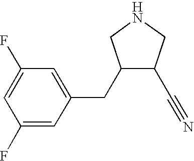

To a solution of rac-1-Benzyl-4-(3,5-difluoro-benzyl)-pyrrolidine-3-carbonitrile (3 g, 9.6 mmol) in DCE (100 mL) is added 1-chloroethyl chloroformate (2.09 mL, 19.2 mmol). The mixture is refluxed overnight and concentrated. The residue is dissolved in MeOH (100 mL) and refluxed for 1 hour. After concentration the residue is purified by column chromatography on silica (2% MeOH in DCM) to afford the title compound as a light yellow syrup. MS (EI+): 223 [M+H]⁺

### F. rac-3-(3,5-Difluoro-benzyl)-4-formyl-pyrrolidine-1-carboxylic acid tert-butyl ester

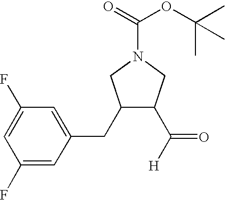

To a solution of rac-4-(3,5-Difluoro-benzyl)-pyrrolidine-3-carbonitrile (780 mg, 2.42 mmol) in THF (50 mL) cooled at −78° C. is added DIBAL-H (4.84 mL of a 1M solution in Hexane, 4.84 mmol) via a syringe pump over 1 hour. The reaction mixture is stirred at −78° C. for another hour and quenched by addition of a few drops of water. The reaction mixture is extracted with EtOAc, washed with water and brine, dried over Na₂SO₄, filtered and concentrated.

Column chromatography on silica of the crude product (40% ethyl acetate in cyclohexane) affords the title compound as a light yellow syrup. MS (EI+): 284 [M+H-tBu]⁺.

### G. rac-3-[(3-tert-Butoxycarbonyl-propylamino)-methyl]-4-(3,5-difluoro-benzyl)-pyrrolidine-1-carboxylic acid tert-butyl ester

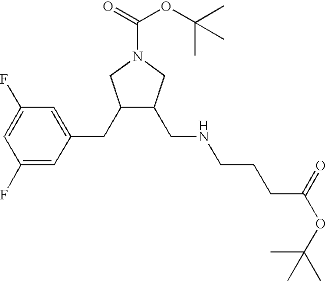

rac-3-(3,5-Difluoro-benzyl)-4-formyl-pyrrolidine-1-carboxylic acid tert-butyl ester (250 mg, 0.77 mmol), H-γ-Abu-OtBu HCl (195 mg, 1 mmol) and Et₃N (160 μL, 1.15 mmol) are mixed in DCE (15 mL) and stirred for 5 min then NaBH(OAc)₃ (244 mg, 1.15 mmol) and AcOH (605 μL, 7.68 mmol) is added. The reaction mixture is stirred at rt for 1 hour and concentrated. Column chromatography on silica of the crude product (50% ethyl acetate in cyclohexane) affords the title compound MS (EI+): 469 [M+H]⁺.

### H. rac-3-{[(3-tert-Butoxycarbonyl-propyl)-(2-oxo-1,2,3,4-tetrahydro-quinoline-4-carbonyl)-amino]-methyl}-4-(3,5-difluoro-benzyl)-pyrrolidine-1-carboxylic acid tert-butyl ester

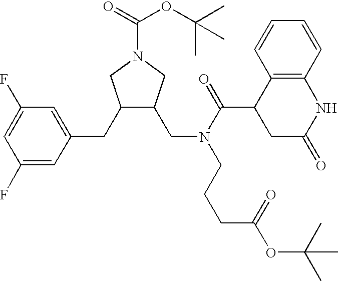

rac-3-[(3-tert-Butoxycarbonyl-propylamino)-methyl]-4-(3,5-difluoro-benzyl)-pyrrolidine-1-carboxylic acid tert-butyl ester (350 mg, 0.75 mmol), 2-Oxo-1,2,3,4-tetrahydro-quinoline-4-carboxylic acid (171 mg, 0.89 mmol), BOP-Cl (294 mg, 1.12 mmol) and Et₃N (519 μL, 3.73 mmol) are mixed in DCM (20 mL). The reaction mixture is refluxed at 40° C. for 30 min and concentrated. Column chromatography on silica of the crude product (5% MeOH in DCM) affords the title compound as a diastereomeric mixture. MS (EI+): 640 [M−H]⁺.

### I. rac-4-[[4-(3,5-Difluoro-benzyl)-pyrrolidin-3-ylmethyl]-(2-oxo-1,2,3,4-tetrahydro-quinoline-4-carbonyl)-amino]-butyric acid

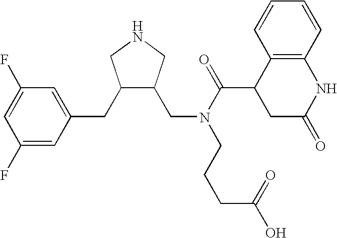

A solution of rac-3-{[(3-tert-Butoxycarbonyl-propyl)-(2-oxo-1,2,3,4-tetrahydro-quinoline-4-carbonyl)-amino]-methyl}-4-(3,5-difluoro-benzyl)-pyrrolidine-1-carboxylic acid tert-butyl ester (350 mg, 0.54 mmol) in MeOH (5 mL) is treated at 0° C. with 4N HCl in dioxane (15 mL). The reaction mixture is stirred for 1 hour at rt and concentrated. Purification on Reverse Phase HPLC affords the title compound as a mixture of diastereoisomers. MS (ESI): 486 [M+H]⁺. t_{R} (HPLC, Nucleosil C18 column, 10-100% CH₃CN/H₂O 5 min, 100% CH₃CN/2.5 min, CH₃CN and H₂O containing 0.1% TFA, flow: 1.0 mL/min): 4.30 and 4.61 min.

### Example 17

### 2-Oxo-1,2,3,4-tetrahydro-quinoline-4-carboxylic acid cyclopropyl-[4-(3,5-difluoro-benzyl)-pyrrolidin-3-yl methyl]-amide

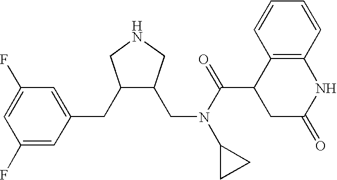

### A. rac-3-Cyclopropylaminomethyl-4-(3,5-difluoro-benzyl)-pyrrolidine-1-carboxylic acid tert-butyl ester

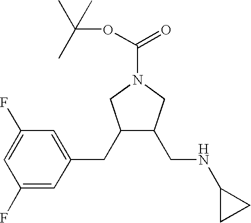

The title compound is prepared analogously as described for the title compound under G in Example 16 (Scheme 5) using rac-3-(3,5-Difluoro-benzyl)-4-formyl-pyrrolidine-1-carboxylic acid tert-butyl ester (200 mg, 0.61 mmol), AcOH (194 μL, 2.46 mmol), NaBH(OAc)₃ (195 mg, 0.92 mmol) and cyclopropylamine (65 μL, 0.92 mmol). MS (EI+): 367 [M+H]⁺

### B. rac-3-{[Cyclopropyl-(2-oxo-1,2,3,4-tetrahydro-quinoline-4-carbonyl)-amino]-methyl}-4-(3,5-difluoro-benzyl)-pyrrolidine-1-carboxylic acid tert-butyl ester

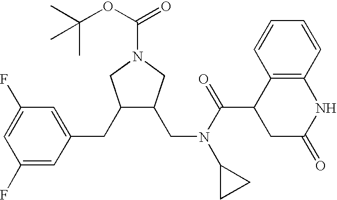

The title compound is prepared analogously as described for the title compound under H in Example 16 (Scheme 5) using rac-3-cyclopropylaminomethyl-4-(3,5-difluoro-benzyl)-pyrrolidine-1-carboxylic acid tert-butyl ester (180 mg, 0.49 mmol) and 2-Oxo-1,2,3,4-tetrahydro-quinoline-4-carboxylic acid (122 mg, 0.64 mmol). MS (EI+): 539 [M+H]⁺

### C. 2-Oxo-1,2,3,4-tetrahydro-quinoline-4-carboxylic acid cyclopropyl-[4-(3,5-difluoro-benzyl)-pyrrolidin-3-yl methyl]-amide

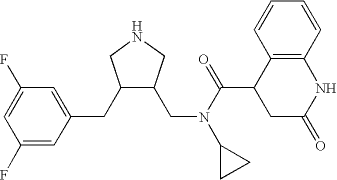

The title compound is prepared analogously as described for the title compound under I in Example 16 (Scheme 5) using rac-3-{[Cyclopropyl-(2-oxo-1,2,3,4-tetrahydro-quinoline-4-carbonyl)-amino]-methyl}-4-(3,5-difluoro-benzyl)-pyrrolidine-1-carboxylic acid tert-butyl ester (115 mg, 0.21 mmol). MS (EI+): 476 [M+H+HCl]⁺ t_{R} (HPLC, Nucleosil C18 column, 10-100% CH₃CN/H₂O 5 min, 100% CH₃CN/2.5 min, CH₃CN and H₂O containing 0.1% TFA, flow: 1.0 mL/min): 4.95 min.

### Example 18

### rac-2-Oxo-1,2,3,4-tetrahydro-quinoline-4-carboxylic acid [4-(3,5-difluoro-benzyl)-pyrrolidin-3-ylmethyl]-pyridin-4-ylmethyl-amide

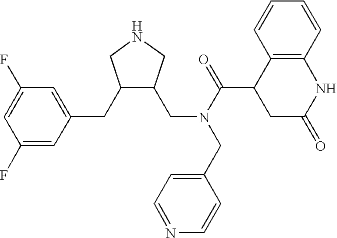

### A. rac-3-(3,5-Difluoro-benzyl)-4-{[(pyridin-4-ylmethyl)-amino]-methyl}-pyrrolidine-1-carboxylic acid tert-butyl ester

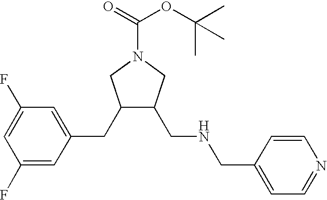

The title compound is prepared analogously as described for the title compound under G in Example 16 (Scheme 5) using rac-3-(3,5-Difluoro-benzyl)-4-formyl-pyrrolidine-1-carboxylic acid tert-butyl ester (200 mg, 0.61 mmol), AcOH (194 L, 2.46 mmol), NaBH(OAc)₃ (195 mg, 0.92 mmol) and 4-picolylamine (93 μL, 0.92 mmol). MS (EI+): 418 [M+H]⁺

### B. rac-3-(3,5-Difluoro-benzyl)-4-{[(2-oxo-1,2,3,4-tetrahydro-quinoline-4-carbonyl)-pyridin-4-ylmethyl-amino]-methyl}-pyrrolidine-1-carboxylic acid tert-butyl ester

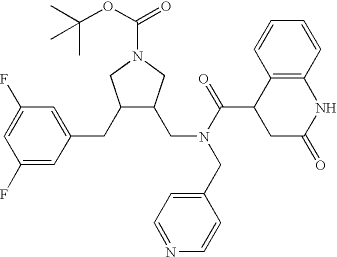

The title compound is prepared analogously as described for the title compound under H in Example 16 (Scheme 5) using rac-3-(3,5-difluoro-benzyl)-4-{[(pyridin-4-ylmethyl)-amino]-methyl}-pyrrolidine-1-carboxylic acid tert-butyl ester (240 mg, 0.58 mmol). MS (EI+): 591 [M+H]⁺

### C. rac-2-Oxo-1,2,3,4-tetrahydro-quinoline-4-carboxylic acid [4-(3,5-difluoro-benzyl)-pyrrolidin-3-ylmethyl]-pyridin-4-ylmethyl-amide

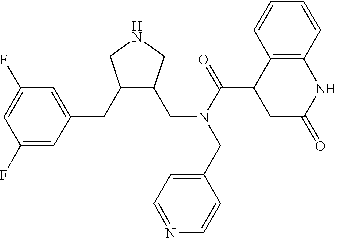

The title compound is prepared analogously as described for the title compound under I in Example 16 (Scheme 5) using rac-3-(3,5-difluoro-benzyl)-4-{[(2-oxo-1,2,3,4-tetrahydro-quinoline-4-carbonyl)-pyridin-4-ylmethyl-amino]-methyl}-pyrrolidine-1-carboxylic acid tert-butyl ester (110 mg, 0.19 mmol). MS (EI+): 491 [M+H]⁺ t_{R} (HPLC, Nucleosil C18 column, 10-100% CH₃CN/H₂O 5 min, 100% CH₃CN/2.5 min, CH₃CN and H₂O containing 0.1% TFA, flow: 1.0 mL/min): 4.32 min.

### Example of Formulation 1: Soft Capsules

5000 soft gelatin capsules, each comprising as active ingredient 0.05 g of any one of the compounds of formula I mentioned in any one of the preceding Examples, are prepared as follows:
1.

Composition

| | |
|---|---|
| Active ingredient | 250 g |
| Lauroglycol | 2 liters |

Preparation process: The pulverized active ingredient is suspended in Lauroglykol® (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

### Example of Formulation 2: Tablets Comprising Compounds of the Formula I

Tablets, comprising, as active ingredient, 100 mg of any one of the compounds of formula I in any one of the preceding Examples are prepared with the following composition, following standard procedures:

Composition

| | |
|---|---|
| Active Ingredient | 100 mg |
| crystalline lactose | 240 mg |
| Avicel | 80 mg |
| PVPPXL | 20 mg |
| Aerosil | 2 mg |
| magnesium stearate | 5 mg |
| | 447 mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinyl-polypyrrolidone, cross-linked (BASF, Germany). Aerosil® is silicon dioxide (Degussa, Germany).

1. A compound of the formula I, 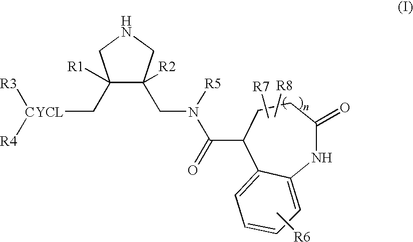
wherein
R¹ and R² are independently of each other hydrogen, C₁-C₇-alkoxy or halogen;
CYCL is aryl or cycloalkyl;
R³ and R⁴ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, amino, mono- or di-(C₁-C₇-alkyl)-amino, C₁-C₇-alkanoylamino, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl, phenyl-C₁-C₇-alkyl- or naphthyl-C₁-C₇-alkyl)carbamoyl and N-mono- or N,N-di(C₁-C₇-alkyl, phenyl-, naphthyl-, phenyl-C₁-C₇-alkyl- or naphthyl-C₁-C₇-alkyl-)sulfamoyl and cyano;
R⁵ is unsubstituted or substituted alkyl, unsubstituted or substituted aryl, substituted or unsubstituted alkenyl, unsubstituted or substituted mono- or bicyclic heterocyclyl, unsubstituted or substituted cycloalkyl, unsubstituted or substituted aryl-alkyl, unsubstituted or substituted mono- or bicyclic heterocyclyl-alkyl or unsubstituted or substituted cycloalkyl-alkyl;
n is 0 or 1;
R⁶, R⁷ and R⁸ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C1-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, amino, mono- or di-(C₁-C₇-alkyl)-amino, C₁-C₇-alkanoylamino, carboxyl, C₁-C₇-alkoxycarbonyl, phenyl- or naphtyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano;
or when R⁷ and R⁸ are both C₁-C₇-alkyl, they may form a C₃-C₇-cycloalkyl ring; in free form or in sat form.

2. The compound according to claim 1 of the formula I, wherein
R¹ and R² are independently of each other hydrogen, C₁-C₇-alkoxy or halogen;
CYCL is aryl or cycloalkyl;
R³ and R⁴ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, amino, mono- or di-(C₁-C₇-alkyl)-amino, C₁-C₇-alkanoylamino, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano;
R⁵ is unsubstituted or substituted alkyl, unsubstituted or substituted aryl, substituted or unsubstituted alkenyl, unsubstituted or substituted mono- or bicyclic heterocyclyl, unsubstituted or substituted cycloalkyl, unsubstituted or substituted aryl-alkyl, unsubstituted or substituted mono- or bicyclic heterocyclyl-alkyl or unsubstituted or substituted cycloalkyl-alkyl;
n is 0 or 1
R⁶, R⁷ and R⁸ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, phenyl- or naphthyloxy, phenyl- or naphthyl-C₁-C₇ alkyloxy, C₁-C₇-alkanoyloxy, nitro, amino, mono- or di-(C₁-C₇-alkyl)-amino, C₁-C₇-alkanoylamino, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl or naphthyl-C₁-C₇-alkyl-)carbamoyl and N-mono- or N,N-di-(C₁-C₇-alkyl-, phenyl-, naphthyl, phenyl-C₁-C₇-alkyl- or naphthyl-C₁-C₇-alkyl-)sulfamoyl and cyano;
or when R⁷ and R⁸ are both C₁-C₇-alkyl, they may form a C₃-C₇-cycloalkyl ring;
where in each case of occurrence above in this claim
unsubstituted or substituted aryl is mono- or polycyclic, especially monocyclic, bicyclic, tricyclic aryl with 6 to 22 carbon atoms, especially phenyl, naphthyl, indenyl or fluorenyl, and is unsubstituted or substituted by one or more, especially one to three, moieties, preferably independently selected from the group consisting of a substitutent of the formula —(C₀-C₇-alkylene)-(X)ᵣ—(C₁-C₇-alkylene-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —O—CO—, —CO—O—, —NV—CO—; —CO—NV—; —NV—SO₂—, —SO₂—NV; —NV—CO—NV—, —NV—CO—C—, —O—CO—NV—, —NV—SO—NV—wherein V is hydrogen or unsubstituted or substituted alkyl as defined below, especially selected from C₁-C₇-alkyl, phenyl, naphthyl, phenyl- or naphthyl-C₁-C₇-alkyl and halo-C₁-C₇-alkyl; e.g. the substituent of said formula is C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl: C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoyloxy, mono- or di-(C₁-C₇-alkyl)-amino, N-mono-C₁-C₇-alkoxy-C₁-C₇-alkylamino, C₁-C₇-alkanoylamino, C₁-C₇-alkylsulfonyamino, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇ alkylcarbamoyl or N-mono- or N,N-di-(C₁-C₇-alkyl)-aminosulfonyl or carboxyl; from C₁-C₇-alkenyl, C₁-C₇-alkinyl, phenyl, naphtyl, cycloalkyl heterocyclyl, especially as defined below for heterocyclyl, preferably selected from pyrrolyl, furanyl, thienyl, pyrimidine-2,4-dione-1-, -3- or -5-yl and benzo[1,3]-dioxolyl, phenyl- or naphthyl- or heterocyclyl-C₁-C₇-alkyl wherein heterocycyl is as defined below preferably selected from pyrrolyl, furanyl, thienyl, pyrimidine-2,4-dione-1-, -3- or -5-yl and benzo[1,3]-dioxolyl, such as benzyl or naphthylmethyl, halo-C₁-C₇-alkyl, such as trifluoromethyl, phenyloxy- or naphthyloxy-C₁-C₇-alkyl, cycloalkyl-C₁-C₇-alkyl, heterocyclyl-C₁-C₇-alkyl, phenyl-C₁-C₇-alkoxy- or naphthyl-C₁-C₇-alkoxy-C₁-C₇-alkyl cycloalkyl-C₁-C₇-alkoxy-C₁-C₇-alkyl, heterocyclyl-C₁-C₇-alkoxy-C₁-C₇-alkyl, di-(naphthyl- or phenyl)-amino-C₁-C₇-alkyl mono- or di-(heterocyclyl-, cycloalkyl-, naphthyl- or phenyl)-amino-C₁-C₇-alkyl, di-(naphthyl- or phenyl-C₁-C₇-alkyl)-amino-C₁-C₇-alkyl, mono- or di-(heterocycyl-, cycloalkyl-, naphthyl- or phenyl-C₁-C₇-alkyl)-amino-C₁-C₇-alkyl, benzoyl- or naphthoylamino-C₁-C₇-alkyl, cycloalkyl-COamino-C₁-C₇-alkyl, heterocyclyl-COamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, cycloalkylsulfonylamino-C₁-C₇-alkyl, heterocyclylsulfonylamino-C₁-C₇-alkyl, phenyl- or naphthyl-C₁-C₇-alkylsulfonylamino-C₁-C₇-alkyl, cycloalkyl-C₁-C₇-alkylsulfonylamino-C₁-C₇-alkyl, heterocyclyl-C₁-C₇-alkylsulfonyamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, cycloalkyl-C₁-C₇-alkoxy, heterocyclyl-C₁-C₇-alkoxy, phenyl- or naphthyloxy, cycloalkyloxy, heterocyclyloxy, phenyl- or naphthyl-C₁-C₇-alkyloxy, cycloalkyl-C₁-C₇-alkyloxy, heterocyclyl-C₁-C₇-alkyloxy, benzoyl- or naphthoyloxy, halo-C₁-C₇-alkylthio, such as trifluoromethylthio, phenyl- or naphthylthio, cycloalkylthio, heterocyclylthio, phenyl- or naphtyl-C₁-C₇-alkylthio, cycloalkyl-C₁-C₇-alkylthio, heterocyclyl-C₁-C₇-alkylthio, benzoyl- or naphthoylthio, nitro, amino, mono- or di-(naphthyl- or phenyl-C₁-C₇ alkyl)-amino, mono- or di-(heterocyclyl-, cycloalkyl-, naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkyl moieties, cycloalkylsulfonylamino, heterocyclylsulfonylamino, phenyl- or naphthyl-C₁-C₇-alkylsulfonylamino, cycloalkyl-C₁-C₇-alkylsulfonylamino, heterocyclyl-C₁-C₇-alkylsulfonylamino, carboxyl, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, N-mono or (N,N—) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, N-mono or (N,N—) di-(C₁-C₇-alkyl)-amino-C₁-C₇-alkoxycarbonyl, carbamoyl, N-mono or N,N-di-(heterocyclyl-, cycloalkyl-, naphthyl- or -phenyl-)-aminocarbonyl, N-mono- or N,N-di-(heterocyclyl-, cycloalkyl-, naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkinylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, cycloalkylsulfinyl, heterocyclylsulfinyl, phenyl- or naphthyl-C₁-C₇-alkylsulfinyl, cycloalkyl-C₁-C₇-alkylsulfinyl, heterocyclyl-C₁-C₇-alkylsulfinyl, sulfonyl, C₁-C₇-alkylsulfonyl, halo C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, N-mono or (N,N—) di-(C₁-C₇-alkyl)-amino-C₁-C₇-alkylsulfinyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkyl moieties, cycloalkylsulfonyl, heterocyclylsulfonyl, phenyl- or naphthyl-C₁-C₇-alkylsulfonyl, cycloalkyl-C₁-C₇-alkylsulfonyl, heterocyclyl-C₁-C₇-alkylsulfonyl, sulfamoyl and N-mono or N,N-di-(C₁-C₇-alkyl, phenyl-, naphthyl, heterocyclyl, cycloalkyl, phenyl-C₁-C₇-alkyl and/or naphthyl-C₁-C₇-alkyl, heterocyclyl-C₁-C₇-alkyl, cycloalkyl-C₁-C₇-alkyl)-aminosulfonyl; unsubstituted or substituted heterocyclyl is a mono- or bicyclic, unsaturated, partially saturated or saturated ring system with preferably 3 to 22 (more preferably 3 to 14) ring atoms and with one or more, preferably one to four, heteroatoms independently selected from nitrogen (N—, —NH— or substituted —NH—), oxygen, sulfur (—S—, S(═O)— or S—(═O)₂—) which is unsubstituted or substituted by one or more, e.g. up to three, substitutents preferably independently selected from the substitutents mentioned above for aryl and from oxo, preferably selected from the following moieties: 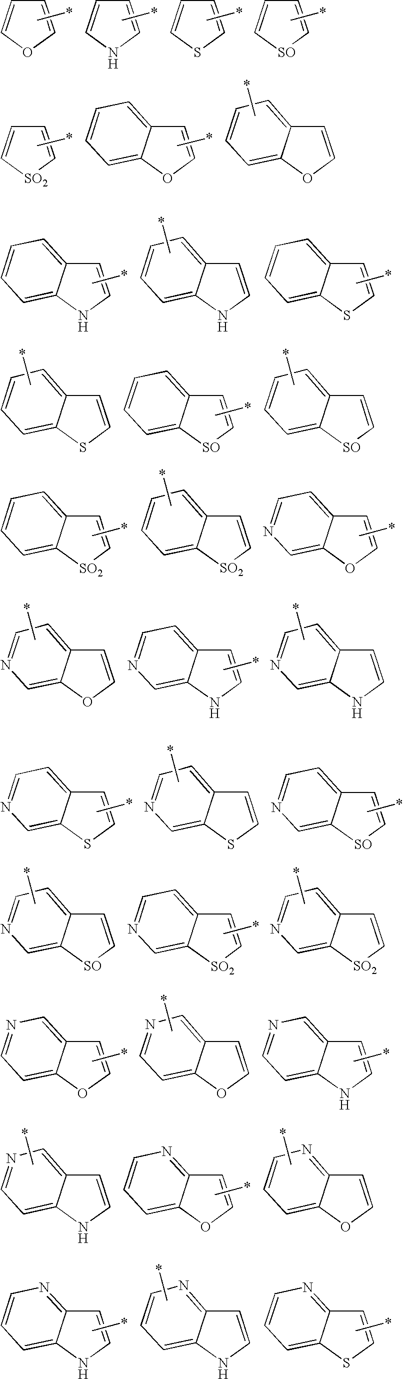 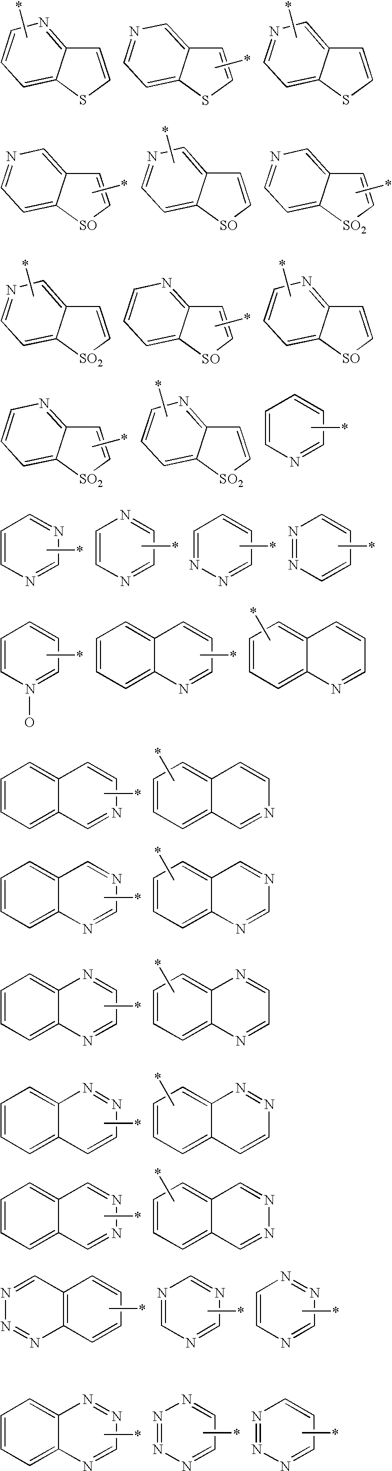 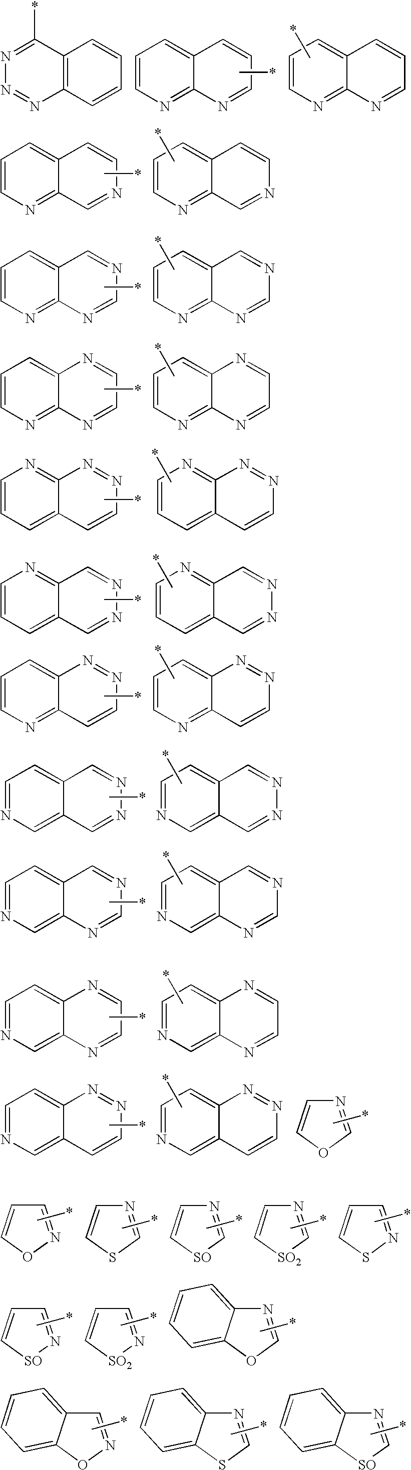 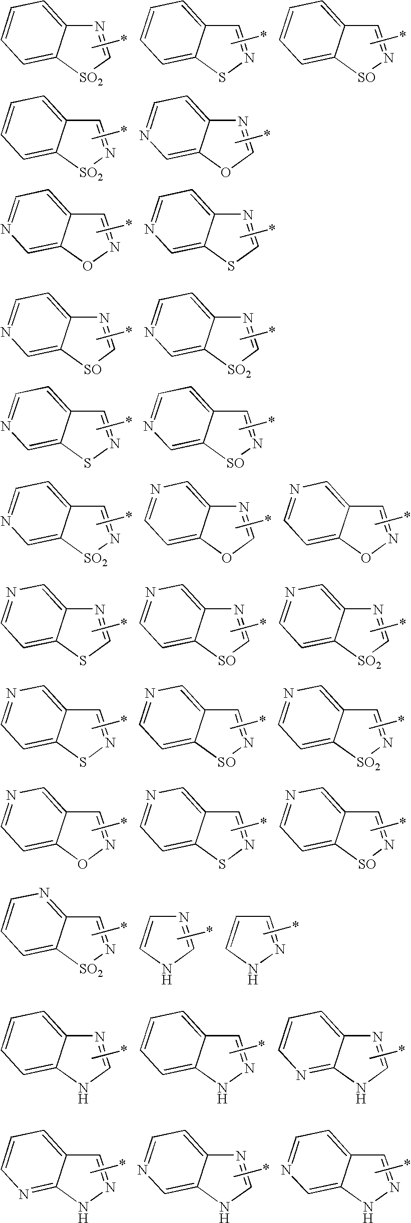 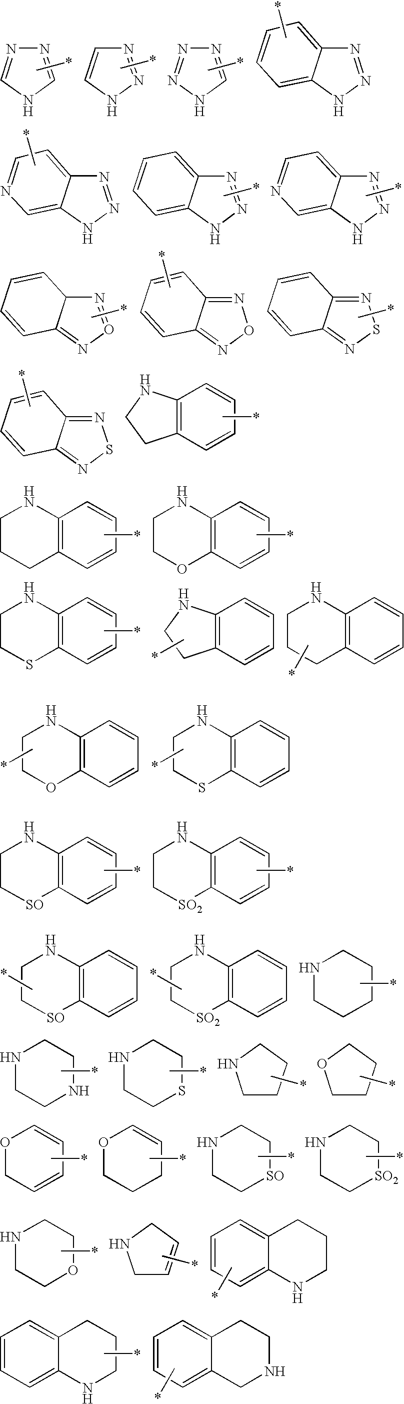 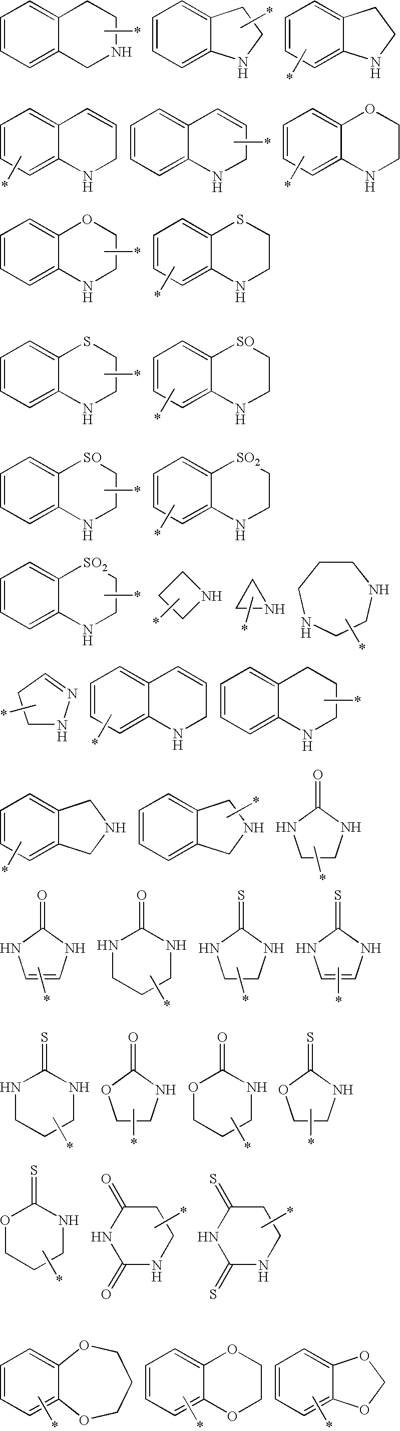
where in each case where an NH is present the bond with the asterisk connecting the respective heterocyclyl moiety to the rest of the molecule the H may be replaced with said bond and/or the H may be replaced by a substituent,
unsubstituted or substituted cycloalkyl is mono- or polycyclic, more preferably monocyclic, C₃-C₁₀-cycloalkyl which may include one or more double (e.g. in cycloalkenyl) and/or triple bonds (e.g. in cycloalkinyl), and is unsubstituted or substituted by one or more, e.g. one to three substitutents preferably independently selected from those mentioned above as substituents for aryl,
in unsubstituted or substituted aryl-alkyl, aryl, which is preferably unsubstituted or substituted by one or more substituents, e.g. one to three substituents independently selected from those mentioned above as substituents for aryl, is preferably as described above for aryl and is bound to alkyl, preferably C₁-C₇ alkyl, either terminally or at any other carbon in the alkyl chain, e.g. at the 1-carbon;
in unsubstituted or substituted heterocyclyl-alkyl, heterocyclyl is as described above and is unsubstituted or substituted by one or more, e.g. up to three, substitutents independently selected from those mentioned above for substituted aryl, and heterocyclyl is bound to alkyl,
preferably C₁-C₇-alkyl, either terminally or at any other carbon in the alkyl chain, e.g. at the 1-carbon;
in unsubstituted or substituted cycloalkyl-alkyl, cycloalkyl is as described above and is unsubstituted or substituted by one or more, e.g. up to three, substitutents independently selected from those mentioned above for substituted aryl, and cycloalkyl is bound to alkyl, preferably C₁-C₇-alkyl, either terminally or at any other carbon in the alkyl chain, e.g. at the 1-carbon;
unsubstituted or substituted alkyl is C₁-C₂₀-alkyl, more preferably C₁-C₇-alkyl, that is straight-chained or branched, which is unsubstituted or substituted by one or more, e.g. up to three moieties selected from unsubstituted or substituted aryl as described above, especially phenyl or naphthyl each of which a unsubstituted or substituted as described above for unsubstituted or substituted aryl, unsubstituted or substituted heterocyclyl as described above, especially pyrrolyl, furanyl, thienyl, pyrimidine-2,4-dione-1-, -2-, -3- or -5-yl and benzo[1,3]dioxolyl, each of which is unsubstituted or substituted as described above for unsubstituted or substituted heterocyclyl; unsubstituted or substituted cycloalkyl as described above, especially cyclopropyl, cyclobutyl, cyclopentyl or cyclohexyl each of which is unsubstituted or substituted as described above for unsubstituted or substituted cycloalkyl; C₂-C₇-alkenyl, C₁-C₇-alkinyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇ alkylthio, halo-C₁-C₇-alkthio, such as trifluoromethylthio, hydroxy-C₁-C₇-alkylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, hydroxy-C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, N-mono- or N,N-di-(alkyl, naphtyl, phenyl, heterocyclyl, cycloalkyl, naphthyl-, heterocyclyclyl-, cycloalkyl- or phenyl-C₁-C₇-alkyl)-aminocarbonyl, cyano, C₁-C₇-alkenylene or -alkinylene, C₁-C₇-alkylenedioxy, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, cycloalkylsulfinyl, heterocyclylsulfinyl, phenyl- or naphthyl-C₁-C₇-alkylsulfinyl, cycloalkyl-C₁-C₇-alkylsufinyl, heterocyclyl-C₁-C₇-alkylsulfinyl, sulfonyl C₁-C₇-alkylsulfonyl, phenyl- or naphthylsulfonyl wherein phenyl or naphthyl is unsubstituted or substituted by one or more, especially one to three, C₁-C₇-alkyl moieties, cycloalkylsulfonyl, heterocyclylsulfonyl, phenyl- or naphthyl-C₁-C₇-alkylsulfonyl, cycloalkyl-C₁-C₇-alkylsulfonyl, heterocycyl-C₁-C₇-alkylsulfonyl, sulfamoyl, N-mono- or N,N-di-(alkyl, naphtyl, phenyl, heterocyclyl, cycloalkyl, naphthyl-, heterocyclyl-, cycloalkyl- or phenyl-C₁-C₇-alkyl)-aminosulfonyl, N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)-aminocarbonylamino and N-mono-, N′-mono-, N,N-di- or N,N,N′-tri-(C₁-C₇-alkyl, hydroxyl-C₁-C₇-alkyl and/or C₁-C₇-alkoxy-C₁-C₇-alkyl)aminosulfonylamino; in substituted or unsubstituted alkylsulfonyl, substituted or unsubstituted alkyl is as defined above for unsubstituted or substituted alkyl;
substituted or unsubstituted alkenyl is as defined above for substituted or unsubstituted alkyl, whereby instead of one or more, preferably one, single bond, a double bond is present;
N-mono- or N,N-di-substituted aminocarbonyl is aminocarbonyl, preferably bound to L=oxy or thio, that is mono- or di-substituted at the nitrogen by one or more moieties selected from unsubstituted or substituted alkyl, unsubstituted or substituted aryl, unsubstituted or substituted heterocyclyl or unsubstituted or substituted cycloalkyl, each of which is defined as above; a preferred example is aryl-C₁-C₇-alkylaminocarbonyl (=aryl-C₁-C₇—NH—C(═O)—), such as benzylaminocarbonyl, bound to L=oxy or further thio; and
N-mono- or N,N-di-substituted aminosulfonyl is sulfamoyl, preferably bound to L=imino or especially oxy, that is mono- or di-substituted at the nitrogen by one or more moieties selected from unsubstituted or substituted alkyl, unsubstituted or substituted aryl, unsubstituted or substituted heterocyclyl or unsubstituted or substituted cycloalkyl, each of which is preferably defined as above; a preferred example is aryl-C₁-C₇-alkylaminosulfonyl (=aryl-C₁-C₇—NH—S(═O)₂—), such as benzylaminosulfonyl, bound to L=oxy or further imino;
in free form or in pharmaceutically acceptable salt form.

3. The compound according to claim 1 of the formula I, wherein R¹ and R² are independently of each other hydrogen or F.

4. The compound according to claim 1 of the formula I, wherein CYCL is phenyl or C₃-C₇-cycloalkyl.

5. The compound according to claim 1 of the formula I, wherein R³ and R⁴ are independently of each other hydrogen, C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl-amino-C₁-C₇-alkyl, C₁-C₇-alkanoylamino-C₁-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano.

6. The compound according to claim 1 of the formula I, wherein R⁵ is unsubstituted or substituted alkyl, unsubstituted or substituted cycloalkyl, unsubstituted or substituted arylalkyl, substituted or unsubstituted alkenyl unsubstituted or substituted mono- or bicyclic heterocyclyl-alkyl or unsubstituted or substituted cycloalkyl-alkyl.

7. The compound according to claim 1 of the formula I, wherein n is 0.

8. The compound according to claim 1 of the formula I, wherein R⁶, R⁷ and R⁸ are independently of each other hydrogen C₁-C₇-alkyl, phenyl- or naphthyl-C₁-C₇-alkyl, halo-C₁-C₇-alkyl, hydroxy-C₁-C₇-alkyl, C₁-C₇-alkoxy-C₁-C₇-alkyl, amino-C₁-C₇-alkyl, mono- or di-(C₁-C₇-alkyl)-amino-C₁-C₇-alkyl, C₁-C₇-alkanoylamino-C1-C₇-alkyl, C₁-C₇-alkylsulfonylamino-C₁-C₇-alkyl, halo, hydroxy, C₁-C₇-alkoxy, C₁-C₇-alkoxy-C₁-C₇-alkoxy, hydroxy-C₁-C₇-alkoxy, nitro, amino, mono- or di-(C₁-C₇-alkyl)-amino, C₁-C₇-alkanoylamino, carboxyl, and cyano, or, when R⁷ and R⁸ are both C₁-C₇-alkyl, they may form a C₃-cycloalkyl ring.

9. The compound according to claim 1 of the formula IA, 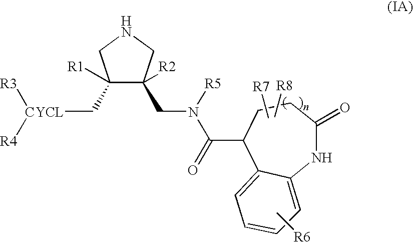
wherein R¹, R², R³, R⁴, R⁵, R⁶, R⁷, R⁸, CYCL and n are as defined, in free form or in pharmaceutically acceptable salt form.

10. The compound according to claim 9 of the formula IA, wherein
R¹ is hydrogen;
R² is hydrogen or F;
CYCL is phenyl or cyclohexyl;
R³ and R⁴ are independently of each other hydrogen, C₁-C₇-alkyl, halo-C₁-C₇-alkyl, hydroxy-C₁-C₇-alkyl, halo, hydroxy, or C₁-C₇-alkoxy;
R⁵ is substituted or unsubstituted C₁-C₇-alkyl, C₃-C₇-cycloalkyl, phenyl-C₁-C₇-alkyl, substituted or unsubstituted C₁-C₇-alkenyl, monocyclic heterocyclyl-C₁-C₇-alkyl or C₁-C₇-cycloalkyl-C₃-C₇-alkyl;
n is 0;
R⁶ is hydrogen, C₁-C₇-alkyl, halo-C₁-C₇-alkyl, hydroxy-C₁-C₇-alkyl, halo, hydroxy, or C₁-C₇-alkoxy;
R⁷ is hydrogen, C₁-C₇-alkyl, halo-C₁-C₇-alkyl, hydroxy-C₁-C₇-alkyl, halo, hydroxy, or C₁-C₇-alkoxy;
R⁸ is hydrogen, C₁-C₇-alkyl, halo-C₁-C₇-alkyl, hydroxy-C₁-C₇-alkyl, halo, hydroxy, or C₁-C₇-alkoxy.

11. The compound according to claim 1 of the formula I, selected from the group of compounds consisting of
2-Oxo-1,2,3,4-tetrahydro-quinoline-4-carboxylic acid ((3S,4S) benzyl-pyrrolidin-3-ylmethyl)-methyl-amide;
2-Oxo-1,2,3,4-tetrahydro-quinoline-4-carboxylic acid ((3S,4S)-4-benzyl-pyrrolidin-3-ylmethyl)-cyclopropyl-amide;
2-Oxo-1,2,3,4-tetrahydro-quinoline-4-carboxylic acid benzyl-((3S,4S)-4-benzyl-pyrrolidin-3-ylmethyl)-amide;
2-Oxo-1,2,3,4-tetrahydro-quinoline-4-carboxylic acid ((3S,4S)₄₈-benzyl-pyrrolidin-3-ylmethyl)-cyclopropylmethyl-amide;
2-Oxo-1,2,3,4-tetrahydro-quinoline-4-carboxylic acid ((3S,4S)-4-benzyl-pyrrolidin-3-ylmethyl)-2-methyl-allyl)-amide;
2-Oxo-1,2,3,4-tetrahydro-quinoline-4-carboxylic acid ((3S,4S)-4-benzyl-pyrrolidin-3-ylmethyl)-isopropyl-amide;
2-Oxo-1,2,3,4-tetrahydro-quinoline-4-carboxylic acid ((3S,4S)-4-benzyl-pyrrolidin-3-ylmethyl)-cyclobutyl-amide;
6-Fluoro-2-oxo-1,2,3,4-tetrahydro-quinoline-4-carboxylic acid ((3S,4S)-4-benzyl-pyrrolidin-3-ylmethyl-cyclopropyl-amide;
4-[((3S,4S)-4-Benzyl-pyrrolidin-3-ylmethyl)-(2-oxo-1,2,3,4-tetrahydro-quinoline-4-carbonyl)-amino]-butyric acid;
2-Oxo-1,2,3,4-tetrahydro-quinoline-4-carboxylic acid ((3S,4S)-4-cyclohexylmethyl-pyrrolidin-3-ylmethyl-methyl-amide;
4-[((3S,4S)-4-cyclohexylmethyl-pyrrolidin-3-ylmethyl-(2-oxo-1,2,3,4-tetrahydro-quinoline-4-carbonyl)-amino]-butyric acid;
2-Oxo-1,2,3,4-tetrahydro-quinoline-4-carboxylic acid ((3S,4S)-4-cyclohexylmethyl-pyrrolidin-3-ylmethyl)-cyclopropyl-amide;
2-Oxo-1,2,3,4-tetrahydro-quinoline-4-carboxylic acid ((3R,4R)-4-benzyl-3-fluoro-pyrrolidin-3-ylmethyl)-cyclopropyl-amide;
(R)-2-Oxo-1,2,3,4-tetrahydro-quinoline-4-carboxylic acid [(3S,4S)-4-(3-bromo-benzyl)-pyrrolidin-3-ylmethyl-]-cyclopropyl-amide;
(R)-2-Oxo-1,2,3,4-tetrahydro-quinoline-4-carboxylic acid [(3S,4S)-4-(2-bromo-benzyl)-pyrrolidin-3-ylmethyl]-cyclopropyl-amide
4-[[4-(3,5-Difluoro-benzyl)pyrrolidin-3-ylmethyl]-(2-oxo-1,2,3,4-tetrahydro-quinoline-4-carbonyl)-amino]-butyric acid;
2-Oxo-1,2,3,4-tetrahydro-quinoline-4-carboxylic acid cyclopropyl-[4-(3,5-difluoro-benzyl)-pyrrolidin-3-yl methyl]-amide; and
rac-2-Oxo-1,2,3,4-tetrahydro-quinoline-4-carboxylic acid [4-(3,5-difluoro-benzyl)-pyrolidin-3-ylmethyl]-pyridin-4-ylmethyl-amide.

12-17. (canceled)

18. A pharmaceutical composition, comprising:
the compound according to claim 1 of the formula I, in free form or in pharmaceutically acceptable salt form, as active ingredient and
at least one pharmaceutically acceptable carrier material.

19. A method for the treatment of a disease of a warm-blooded animal, that depends on the activity of beta-secretase and the generation of beta-amyloid and/or the subsequent aggregation into oligomers and fibrils, comprising:
administering to a warm-blooded animal in need of such treatment a pharmaceutically effective amount of the compound according to claim 1 of the formula I, in free form or in pharmaceutically acceptable salt form.

20. The method according to claim 19 wherein the disease is neurodegenerative diseases.

21. A pharmaceutical composition, comprising:
the compound according to claim 9 of the formula IA, in free form or in pharmaceutically acceptable salt form, as active ingredient and
at least one pharmaceutically acceptable carrier material.

22. A method for the treatment of a disease of a warm-blooded animal that depends on the activity of beta-secretase and the generation of beta-amyloid and/or the subsequent aggregation into oligomers and fibrils, comprising:
administering to a warm-blooded animal in need of such treatment a pharmaceutically effective amount of the compound according to claim 9 of the formula IA, in free form or in pharmaceutically acceptable salt form.

23. The method according to claim 22, wherein the disease is neurodegenerative diseases.

24. The method according to claim 20, wherein the neurodegenerative diseases are Alzheimer's disease, Down's syndrome, memory and cognitive impairment, dementia, amyloid neuropathies, brain inflammation, nerve and brain trauma, vascular amyloidosis, or cerebral haemorrhage with amyloidosis.

25. The method according to claim 23, wherein the neurodegenerative diseases are Alzheimer's disease, Down's syndrome, memory and cognitive impairment, dementia, amyloid neuropathies, brain inflammation, nerve and brain trauma, vascular amyloidosis, or cerebral haemorrhage with amyloidosis.
